(19) United States
(12) Patent Application Publication
(10) Pub. No.: US 2024/0180939 A1
(43) Pub. Date: Jun. 6, 2024

(54) IMMUNOMODULATORY OLIGOSACCHARIDES FOR THE ENHANCEMENT OF ANTI-TUMOR EFFICACY OF IMMUNO-ONCOLOGY AGENTS

(71) Applicant: Intrinsic Medicine, Inc., Seattle, WA (US)

(72) Inventors: Alexander Martinez, Des Moines, WA (US); Jason Ferrone, San Diego, CA (US); Emil Chuang, Park City, UT (US)

(21) Appl. No.: 18/212,223

(22) Filed: Jun. 21, 2023

Related U.S. Application Data
(63) Continuation of application No. PCT/US2021/064514, filed on Dec. 21, 2021.
(60) Provisional application No. 63/129,064, filed on Dec. 22, 2020.

(51) Int. Cl. A61K 31/702; A61K 45/06

(52) U.S. Cl. CPC A61K 31/702; A61K 45/06

(57) ABSTRACT

The disclosure provides for methods of enhancing anti-tumor therapies with immunomodulatory human milk oligosaccharides.

### RELATED APPLICATIONS

This application is a continuation of International Application No. PCT/US21/64514, which designated the United States and was filed on Dec. 21, 2021, published in English which claims the benefit of U.S. Provisional Application No. 63/129,064 filed Dec. 22, 2020. The entire contents of the above-referenced applications are incorporated by reference herein.

### BACKGROUND OF THE INVENTION

Immune checkpoint proteins are molecules on the surface of immune cells that control T-cell activation and prevent the immune system from targeting healthy cells. Certain cancer and tumor cells take advantage of these immune checkpoint molecules to evade the immune system. For example, the checkpoint protein PD-1 on immune cells binds to PD-L1 on the cancer cell surface and this binding inhibits immune cell targeting. Immune checkpoint inhibitors block immune checkpoint molecules and allow the immune system, and T-cells specifically, to target cancer and tumor cells as well as the cancer immune environment (Robert, 2019. Nature Communications 11:3801). Various immune checkpoint inhibitors have been developed that target different checkpoint proteins, including programmed cell death receptor 1 (PD-1)/PD-1 ligand-1 (PDL1) as well as cytotoxic T-lymphocyte-associated protein-4 (CTLA-4). For example, ipilimumab, an anti-CTLA-4 monoclonal antibody has been approved for the treatment of advanced melanoma. Nivolumab, an anti-PD-1 monoclonal antibody, has also been approved for the treatment of patients with certain advanced cancers.

Despite these advances, there remains a need in the art to improve cancer therapy, and specifically cancer immunotherapy.

### SUMMARY OF THE INVENTION

The invention is directed to compositions comprising an immune checkpoint inhibitor and a human milk oligosaccharide (HMO). The invention additionally encompasses methods of treating cancer comprising co-administering to a patient a human milk oligosaccharide and an immune checkpoint inhibitor.

The human milk oligosaccharide can be selected from the group consisting of 2′fucosyllactose (2′FL), 3′ sialyllactose (3′SL), and 6′ sialyllactose (6′SL), or a combination of any of thereof.

The immune checkpoint inhibitor can, for example, be an inhibitor of PD-1, PD-L1, PD-L2, PD-L3, PD-L4, CTLA-4, LAG3, B7-H3, B7-H4, KIR or TIM3. In yet additional aspects, the immune checkpoint inhibitor is an antibody. In yet further aspects, the antibody is selected from the group consisting of anti-PD-1, anti-PD-L1, anti-CTLA-4, anti-IDO, anti-KIR, anti-TIM-3, anti-LAG-3, anti-CD39, anti-CD73, anti-ICOS, anti-OX40, anti-Tim-3, anti-Vista, anti-BTLA, anti-TDO, anti-TIGIT-antibody, a-CD3-APC, (X-CD3-APC-H7, a-CD4-ECD, a-CD4-PB, a-CD8-PE-Cy7, a-CD-8-PerCP-Cy5.5, a-CD 11 c-APC, a-CD 11 b-PE-Cy7, a-CDII b-AF700, a-CD 14-FITC, a-CD16-PB, a-CD 19-AF780, a-CD 19-AF700, a-CD20-PO, a-CD25-PE-Cy7, a-CD40-APC, a-CD45-Biotin, Streptavidin-BV605, a-CD62L-ECD, a-CD69-APC-Cy7, a-CD80-FITC, a-CD83-Biotin, Streptavidin-PE-Cy7, a-CD86-PE-Cy7, a-CD86-PE, a-CD123-PE, a-CD 154-PE, a-CD161-PE, a-CTLA4-PE-Cy7, a-FoxP3-AF488 (clone 259D), IgG1-isotype-AF488, a-ICOS (CD278)-PE, a-HLA-A2-PE, a-HLA-DR-PB, a-HLA-DR-PerCPCy5.5, and a-PD1-APC (“a-” is an abbreviation for “anti-”), and antibodies against VISTA, co-stimulatory molecule OX40, and CD 137. In certain aspects, the antibody is an anti-PD-1 antibody, an anti-PD-L1 antibody, an anti-PD-L2 antibody, and/or an anti-CTLA-4 antibody.

The composition can further comprise a pharmaceutically acceptable excipient or carrier.

In certain aspects, the composition further comprises a chemotherapeutic agent.

In other aspects, the method of treating cancer can further comprise administration of a chemotherapeutic agent.

### DETAILED DESCRIPTION OF THE INVENTION

As used herein and in the appended claims, the singular forms “a,” “an,” and “the” include plural referents unless the context clearly dictates otherwise. Thus, for example, reference to “an oligosaccharide” includes a plurality of such oligosaccharides and reference to “the therapeutic agent” includes reference to one or more therapeutic agents and equivalents thereof known to those skilled in the art, and so forth.

Also, the use of “or” means “and/or” unless stated otherwise. Similarly, “comprise,” “comprises,” “comprising” “include,” “includes,” and “including” are interchangeable and not intended to be limiting.

It is to be further understood that where descriptions of various embodiments use the term “comprising,” those skilled in the art would understand that in some specific instances, an embodiment can be alternatively described using language “consisting essentially of” or “consisting of.”

Unless defined otherwise, all technical and scientific terms used herein have the same meaning as commonly understood to one of ordinary skill in the art to which this disclosure belongs.

Although many methods and reagents are similar or equivalent to those described herein, the exemplary methods and materials are disclosed herein. All publications mentioned herein are incorporated herein by reference in full for the purpose of describing and disclosing the methodologies, which might be used in connection with the description herein. Moreover, for terms expressly defined in this disclosure, the definition of the term as expressly provided in this disclosure will control in all respects, even if the term has been given a different meaning in a publication, dictionary, treatise, and the like.

The term “about” as used herein, in reference to a numerical value or range, allows for a degree of variability in the value or range, for example, within 10%, within 5%, or within 4%, or within 2% of the value or range.

The term “pharmaceutically acceptable carrier,” “pharmaceutically acceptable excipient,” “physiologically acceptable carrier,” or “physiologically acceptable excipient” as used herein, refers to a pharmaceutically-acceptable material, composition, or vehicle, such as a liquid or solid filler, diluent, excipient, solvent, or encapsulating material. Each component must be “pharmaceutically acceptable” in the sense of being compatible with the other ingredients of a pharmaceutical formulation. It must also be suitable for use in contact with the tissue or organ of humans and animals without excessive toxicity, irritation, allergic response, immunogenecity, or other problems or complications, commensurate with a reasonable benefit/risk ratio. Examples of “pharmaceutically acceptable carriers” and “pharmaceutically acceptable excipients” can be found in the following, Remington: The Science and Practice of Pharmacy, 21st Edition; Lippincott Williams & Wilkins: Philadelphia, Pa., 2005; Handbook of Pharmaceutical Excipients, 5th Edition; Rowe et al., Eds., The Pharmaceutical Press and the American Pharmaceutical Association: 2005; and Handbook of Pharmaceutical Additives, 3rd Edition; Ash and Ash Eds., Gower Publishing Company: 2007; Pharmaceutical Preformulation and Formulation, Gibson Ed., CRC Press LLC: Boca Raton, Fla., 2004.

The term “release controlling excipient” as used herein, refers to an excipient whose primary function is to modify the duration or place of release of the active substance from a dosage form as compared with a conventional immediate release dosage form. The term “non-release controlling excipient” as used herein, refers to an excipient whose primary function do not include modifying the duration or place of release of the active substance from a dosage form as compared with a conventional immediate release dosage form.

The term “subject” as used herein, refers to an animal, including, but not limited to, a primate (e.g., human, monkey, chimpanzee, gorilla, and the like), rodents (e.g., rats, mice, gerbils, hamsters, ferrets, and the like), lagomorphs, swine (e.g., pig, miniature pig), equine, canine, feline, and the like. The terms “subject” and “patient” are used interchangeably herein. For example, a mammalian subject can refer to a human patient. In preferred aspects, the subject is a human patient.

The term “substantially pure” as used herein in reference to a given oligosaccharide means that the oligosaccharide is substantially free from other biological macromolecules. The substantially pure oligosaccharide is at least 75% (e.g., at least 80, 85, 95, or 99%) pure by dry weight. Purity can be measured by any appropriate standard method, for example, by column chromatography, polyacrylamide gel electrophoresis, or HPLC analysis.

The terms “treat”, “treating” and “treatment,” as used herein, refers to ameliorating symptoms associated with a disease or disorder (e.g., cancer), including inhibiting the progress of the disease or disorder (e.g., cancer), reducing the severity of the disease or disorder (e.g., cancer), preventing or delaying the onset of the disease or disorder symptoms, and/or lessening the severity or frequency of symptoms of the disease or disorder.

The methods described herein comprise co-administration of a human milk oligosaccharide and an immune checkpoint inhibitor. The invention can also entail co-administration of a chemotherapeutic agent with the oligosaccharide and the immune checkpoint inhibitor. As used herein, “co-administration” means administration of at least two therapeutically active drugs or compositions (e.g., administration of the human milk oligosaccharide and an immune checkpoint inhibitor, or pharmaceutical compositions thereof), at different times or simultaneously or substantially simultaneously in either separate formulation or the same formulation/composition. When the at least two therapeutic agents are administered at different times, their administration can be separated by minutes, hours, days, weeks, or months, and/or be administered as part of the same treatment regimen.

An “effective amount” or a “therapeutically effective amount” of an active agent or drug as described herein refers to an amount of the agent, alone or in combination with another active agent, that is sufficient to achieve a specific effect or result, and/or treats the disease or condition and/or the symptoms therefore, for example, alleviating, in whole or in part, symptoms associated with the disorder or condition, or halts or slows further progression or worsening of those symptoms, or prevents or provides prophylaxis for the disorder or condition. For example, an “effective amount” of a human milk oligosaccharide, as described herein, encompasses an amount that, in combination with an immune checkpoint inhibitor, is effective to treat cancer. An “effective amount” of an immune checkpoint inhibitor is an amount that alone, or in combination with an oligosaccharide and/or a chemotherapeutic agent, is effective to treat cancer.

The terms “active agent,” “drug,” “therapeutic agent,” are used interchangeably herein and refer to an agent administered as part of a method of treatment, alone or in combination with one or more pharmaceutically acceptable excipients and/or carriers, to a subject for treating, preventing, or ameliorating one or more symptoms of a disorder. The terms “active agent,” “therapeutic agent,” and “drug” as used herein includes, but are not limited to, oligosaccharides, immune checkpoint inhibitors and/or a chemotherapeutic agent.

The term “disorder” as used herein is intended to be generally synonymous, and is used interchangeably with, the terms “disease,” “syndrome” and “condition” (as in medical condition), in that all reflect an abnormal condition of the body or of one of its parts that impairs normal functioning and is typically manifested by distinguishing signs and symptoms.

The invention encompasses methods of treating cancer in a patient in need thereof. Non-limiting examples of cancer include breast cancer, inflammatory breast cancer, ductal carcinoma, lobular carcinoma, colon cancer, pancreatic cancer, insulinomas, adenocarcinoma, ductal adenocarcinoma, adenosquamous carcinoma, acinar cell carcinoma, glucagonoma, skin cancer, melanoma, metastatic melanoma, lung cancer, small cell lung cancer, non-small cell lung cancer, squamous cell carcinoma, adenocarcinoma, large cell carcinoma, brain (gliomas), glioblastomas, astrocytomas, glioblastoma multiforme, Bannayan-Zonana syndrome, Cowden disease, Lhermitte-Duclos disease, Wilm's tumor, Ewing's sarcoma, Rhabdomyosarcoma, ependymoma, medulloblastoma, head and neck, kidney, liver, melanoma, ovarian, pancreatic, adenocarcinoma, ductal adenocarcinoma, adenosquamous carcinoma, acinar cell carcinoma, glucagonoma, insulinoma, prostate, sarcoma, osteosarcoma, giant cell tumor of bone, thyroid, lymphoblastic T cell leukemia, chronic myelogenous leukemia, chronic lymphocytic leukemia, hairy-cell leukemia, acute lymphoblastic leukemia, acute myelogenous leukemia, chronic neutrophilic leukemia, acute lymphoblastic T cell leukemia, plasmacytoma, Immunoblastic large cell leukemia, mantle cell leukemia, multiple myeloma, megakaryoblastic leukemia, multiple myeloma, acute megakaryocyte leukemia, promyelocytic leukemia, erythroleukemia, malignant lymphoma, Hodgkins lymphoma, non-Hodgkins lymphoma, lymphoblastic T cell lymphoma, Burkitt's lymphoma, follicular lymphoma, neuroblastoma, bladder cancer, urothelial cancer, vulval cancer, cervical cancer, endometrial cancer, renal cancer, mesothelioma, esophageal cancer, salivary gland cancer, hepatocellular cancer, gastric cancer, nasopharyngeal cancer, buccal cancer, cancer of the mouth, GIST (gastrointestinal stromal tumor), neuroendocrine cancers and testicular cancer. The term “cancer” includes solid tumors and metastases.

A chemotherapeutic agent is a drug used for chemotherapy. When the chemotherapeutic agent is used in combination with an immune checkpoint inhibitor as described herein, the chemotherapeutic agent is other than an immune checkpoint inhibitor and can be referred to herein as “an additional chemotherapeutic agent.” Chemotherapeutic agents include, but are not limited to, ABVD; AC; ACE; Abiraterone (Zytiga); Abraxane; Abstral; Actinomycin D; Actiq; Adriamycin; Afatinib (Giotrif); Afinitor; Aflibercept (Zaltrap); Aldara; Aldesleukin (IL-2, Proleukin or interleukin 2); Alemtuzumab (MabCampath); Alkeran; Amsacrine (Amsidine, m-AMSA); Amsidine; Anastrozole (Arimidex); Ara C; Aredia; Arimidex; Aromasin; Arsenic trioxide (Trisenox, ATO); Asparaginase (Crisantaspase, Erwinase); Axitinib (Inlyta); Azacitidine (Vidaza); BEACOPP; BEAM; Bendamustine (Levact); Bevacizumab (Avastin); Bexarotene (Targretin); Bicalutamide (Casodex); Bleomycin; Bleomycin, etoposide and platinum (BEP); Bortezomib (Velcade); Bosulif; Bosutinib (Bosulif); Brentuximab (Adcetris); Brufen; Buserelin (Suprefact); Busilvex; Busulfan (Myleran, Busilvex); CAPE-OX; CAPDX; CAV; CAVE; CCNU; CHOP; CMF; CMV; CVP; Cabazitaxel (Jevtana); Cabozantinib (Cometriq); Caelyx; Calpol; Campto; Capecitabine (Xeloda); Caprelsa; Carbo MV; CarboTaxol; Carboplatin; Carboplatin and etoposide; Carboplatin and paclitaxel; Carmustine (BCNU, Gliadel); Casodex; Ceritinib (Zykadia); Cerubidin; Cetuximab (Erbitux); ChIVPP; Chlorambucil (Leukeran); Cisplatin; Cisplatin and Teysuno; Cisplatin and capecitabine (CX); Cisplatin, etoposide and ifosfamide (PEI); Cisplatin, fluorouracil (5-FU) and trastuzumab; Cladribine (Leustat, LITAK); Clasteon; Clofarabine (Evoltra); Co-codamol (Kapake, Solpadol, Tylex); Cometriq; Cosmegen; Crisantaspase; Crizotinib (Xalkori); Cyclophosphamide; Cyclophosphamide, thalidomide and dexamethasone (CTD); Cyprostat; Cyproterone acetate (Cyprostat); Cytarabine (Ara C, cytosine arabinoside); Cytarabine into spinal fluid; Cytosine arabinoside; DHAP; DTIC; Dabrafenib (Tafinlar); Dacarbazine (DTIC); Dacogen; Dactinomycin (actinomycin D, Cosmegen); Dasatinib (Sprycel); Daunorubicin; De Gramont; Decapeptyl SR; Decitabine (Dacogen); Degarelix (Firmagon); Denosumab (Prolia, Xgeva); Depocyte; Dexamethasone; Diamorphine; Disodium pamidronate; Disprol; Docetaxel (Taxotere); Docetaxel, cisplatin and fluorouracil (TPF); Doxifos; Doxil; Doxorubicin (Adriamycin); Doxorubicin and ifosfamide (Doxifos); Drogenil; Durogesic; EC; ECF; EOF; EOX; EP (Etoposide and cisplatin); ESHAP; Effentora; Efudix; Eldisine; Eloxatin; Enzalutamide; Epirubicin (Pharmorubicin); Epirubicin cisplatin and capecitabine (ECX); Epirubicin, carboplatin and capecitabine (ECarboX); Eposin; Erbitux; Eribulin (Halaven); Erlotinib (Tarceva); Erwinase; Estracyt; Etopophos; Etoposide (Eposin, Etopophos, Vepesid); Everolimus (Afinitor); Evoltra; Exemestane (Aromasin); FAD; FEC; FEC-T chemotherapy; FMD; FOLFIRINOX; FOLFOX; Faslodex; Femara; Fentanyl; Firmagon; Fludara; Fludarabine (Fludara); Fludarabine, cyclophosphamide and rituximab (FCR); Fluorouracil (5FU); Flutamide; Folinic acid, fluorouracil and irinotecan (FOLFIRI); Fulvestrant (faslodex); G-CSF; Gefitinib (Iressa); GemCarbo (gemcitabine and carboplatin); GemTaxol; Gemcitabine (Gemzar); Gemcitabine and capecitabine (GemCap); Gemcitabine and cisplatin (GC); Gemcitabine and paclitaxel (GemTaxol); Gemzar; Giotrif; Gliadel; Glivec; Gonapeptyl Depot; Goserelin (Zoladex); Goserelin (Zoladex, Novgos); Granulocyte colony stimulating factor (G-CSF); Halaven; Herceptin; Hycamtin; Hydrea; Hydroxycarbamide (Hydrea); Hydroxyurea; I-DEX; ICE; IL-2; IPE; Ibandronic acid; Ibritumomab (Zevalin); Ibrutinib (Imbruvica); Ibuprofen (Brufen, Nurofen); Iclusig; Idarubicin (Zavedos); Idarubicin and dexamethasone; Idelalisib (Zydelig); Ifosfamide (Mitoxana); Imatinib (Glivec); Imiquimod cream (Aldara); Imnovid; Instanyl; Interferon (Intron A); Interleukin; Intron A; Ipilimumab (Yervoy); Iressa; Irinotecan (Campto); Irinotecan and capecitabine (Xeliri); Irinotecan de Gramont; Irinotecan modified de Gramont; Javlor; Jevtana; Kadcyla; Kapake; Keytruda; Lanreotide (Somatuline); Lanvis; Lapatinib (Tyverb); Lenalidomide (Revlimid); Letrozole (Femara); Leukeran; Leuprorelin (Prostap, Lutrate); Leustat; Levact; Liposomal doxorubicin; Litak; Lomustine (CCNU); Lynparza; Lysodren; MIC; MMM; MPT; MST Continus; MVAC; MVP; MabCampath; Mabthera; Maxtrex; Medroxyprogesterone acetate (Provera); Megace; Megestrol acetate (Megace); Melphalan (Alkeran); Mepact; Mercaptopurine (Xaluprine); Methotrexate; Methyl prednisolone; Mifamurtide (Mepact); Mitomycin C; Mitotane; Mitoxana; Mitoxantrone (Mitozantrone); Morphgesic SR; Morphine; Myleran; Myocet; Nab-paclitaxel; Nab-paclitaxel (Abraxane); Navelbine; Nelarabine (Atriance); Nexavar; Nilotinib (Tasigna); Nintedanib (Vargatef); Nipent; Nivolumab (Opdivo); Novgos; Nurofen; Obinutuzumab (Gazyvaro); Octreotide; Ofatumumab (Arzerra); Olaparib (Lynparza); Oncovin; Onkotrone; Opdivo; Oramorph; Oxaliplatin (Eloxatin); Oxaliplatin and capecitabine (Xelox); PAD; PC (paclitaxel and carboplatin, CarboTaxol); PE; PMitCEBO; POMB/ACE; Paclitaxel (Taxol); Paclitaxel and carboplatin; Pamidronate; Panadol; Panitumumab (Vectibix); Paracetamol; Pazopanib (Votrient); Pembrolizumab (Keytruda); Pemetrexed (Alimta); Pemetrexed and carboplatin; Pemetrexed and cisplatin; Pentostatin (Nipent); Perjeta; Pertuzumab (Perjeta); Pixantrone (Pixuvri); Pixuvri; Pomalidomide (Imnovid); Ponatinib; Potactasol; Prednisolone; Procarbazine; Procarbazine, lomustine and vincristine (PCV); Proleukin; Prolia; Prostap; Provera; Purinethol; R-CHOP; R-CVP; R-DHAP; R-ESHAP; R-GCVP; RICE; Raloxifene; Raltitrexed (Tomudex); Regorafenib (Stivarga); Revlimid; Rituximab (Mabthera); Sevredol; Sodium clodronate (Bonefos, Clasteon, Loron); Solpadol; Sorafenib (Nexavar); Steroids (dexamethasone, prednisolone, methylprednisolone); Streptozocin (Zanosar); Sunitinib (Sutent); Sutent; TAC; TIP; Tafinlar; Tamoxifen; Tarceva; Targretin; Tasigna; Taxol; Taxotere; Taxotere and cyclophosphamide (TC); Temodal; Temozolomide (Temodal); Temsirolimus; Tepadina; Teysuno; Thalidomide; Thiotepa (Tepadina); Tioguanine (thioguanine, 6-TG, 6-tioguanine); Tomudex; Topotecan (Hycamtin, Potactasol); Torisel; Trabectedin (Yondelis); Trastuzumab (Herceptin); Trastuzumab emtansine (Kadcyla); Treosulfan; Tretinoin (Vesanoid, ATRA); Triptorelin; Trisenox; Tylex; Tyverb; VIDE; Vandetanib (Caprelsa); Vargatef; VeIP; Vectibix; Velbe; Velcade; Vemurafenib (Zelboraf); Vepesid; Vesanoid; Vidaza; Vinblastine (Velbe); Vincristine; Vincristine, actinomycin D (dactinomycin) and cyclophosphamide (VAC); Vincristine, actinomycin and ifosfamide (VAI); Vincristine, doxorubicin and dexamethasone (VAD); Vindesine (Eldisine); Vinflunine (Javlor); Vinorelbine (Navelbine); Vismodegib (Erivedge); Votrient; XELOX; Xalkori; Xeloda; Xgeva; Xtandi; Yervoy; Yondelis; Z-DEX; Zaltrap; Zanosar; Zavedos; Zelboraf; Zevalin; Zoladex (breast cancer); Zoladex (prostate cancer); Zoledronic acid (Zometa); Zometa; Zomorph; Zydelig; and Zytiga.

The invention encompasses methods of treating cancer in a patient or subject in need thereof comprising co-administering an effective amount of a human milk oligosaccharide and an effective amount of an immune checkpoint inhibitor. The invention also encompasses pharmaceutical compositions comprising an immune checkpoint inhibitor and a human milk oligosaccharide, for example, in effective amounts; the pharmaceutical composition can optionally comprise a pharmaceutically acceptable carrier or excipient.

Human milk oligosaccharides (HMOs) are separated into different classes including, for example, sialylated human milk oligosaccharides and fucosylated oligosaccharides. Sialyllactose is a class of human milk oligosaccharides (HMOs) that appear in two different forms in human milk. These two forms are 3′-sialyllactose (3′-SL) and 6′-sialyllactose (6′-SL):

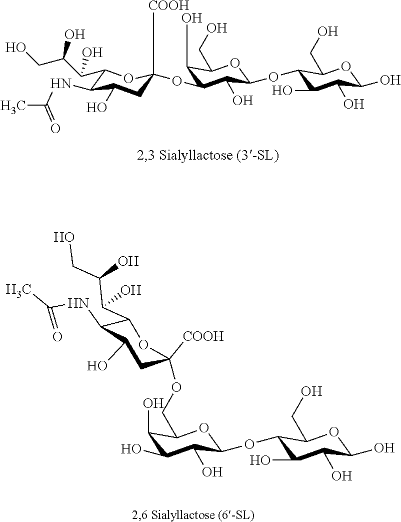

The terms “3′-SL” and “3′SL” are used interchangeably herein. Similarly, the terms “6′-SL” and “6′SL” are used interchangeably herein. Sialyllactoses have been shown to modulate acute and chronic immune responses in both murine and human derived macrophages stimulated with LPS and various pro-inflammatory cytokines. Both 3′SL and 6′SL have shown reductions in interleukin (IL)-1p, IL-2, IL-4, IL-6, IL-12, interferon (IFN) 7 or TNF-α in vitro, with 3′-SL exhibiting more significant reductions. In addition, 3′SL has been shown to reduce other key target proteins, including PDL1, COX2 and select chemokines, such as CCL2 (also known as monocyte chemoattractant protein 1 (MCP1)) and CCL5. In vivo data in mouse models of rheumatoid arthritis, which include an LPS challenge, sialyllactose has shown benefit in clinical assessments of disease when administered orally.

Fucosylated oligosaccharides are a class of human milk oligosaccharides (HMOs) that have been associated with the production of anti-inflammatory short-chain fatty acids. Fucosylated oligosaccharides include, for example, 2′-fucosyllactose, 3-fucosyllactose, difucosyllactose, lacto-N-fucopentaoses (that is to say lacto-N-fucopentaose I, lacto-N-fucopentaose II, lacto-N-fucopentaose III and lacto-N-fucopentaose V), lacto-N-difucohexaose I, fucosyllacto-N-hexaose, Difucosyllacto-N-hexaose I and Difucosyllacto-N-neohexaose II. In certain aspects, the fucosylated non-digestible oligosaccharide is 2′-fucosyllactose (2′-FL). In certain aspects, the fucosylated oligosaccharide is 2′-fucosyllactose (2′-FL), 3-fucosyllactose (3′-FL), difucosyllactose (DFL). In yet further aspects, the fucosylated oligosaccharide is 2′-FL.

Fucosyllactose (FL) is a fucosylated non-digestible oligosaccharide present in human milk but not in cow milk. The primary fucosylated HMO is 2′-fuscosyllactose or 2′FL. It consists of three monosaccharide units, fucose, galactose and glucose linked together. Lactose is a galactose unit linked to a glucose unit via a beta 1,4 linkage. A fucose unit is linked to a galactose unit of a lactose molecule via an alpha 1,2 linkage (2′-fucosyllactose, 2′-FL) or via an alpha 1,3 linkage to the glucose unit of a lactose (3-Fucosyllactose, 3-FL). 2-′FL has the chemical structure shown below:

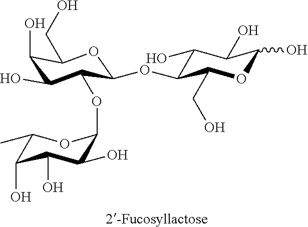

The terms 2′-fucosyllactose or “2′-FL” and “2′FL” are used interchangeably herein. 2′-fucosyllactose has been granted generally regarded as safe (GRAS) status in the U.S. and is regarded by the Europe Food Safety Authority as safe for infant and follow-on formula. 2′-FL has been shown to have many beneficial properties, such as improving of gut health through modulation of the gut microbiome as well as reduction of local gut inflammation in models of necrotizing enterocolitis and other inflammatory bowel diseases. In addition, 2′-FL has been shown to have positive effects on gut epithelial barrier function and also independent anti-inflammatory effects through the reduction in TNFα and IL-8.

Without wishing to be bound by theory, it is believed that human milk oligosaccharides' effectiveness and activity in the treatment of cancer and/or in combination with an immune checkpoint inhibitor as described herein is at least partially due to their role in restoring homeostasis. Normal intestinal flora generate and synthesize various immunomodulatory compounds and metabolites such as short-chain fatty acids (SCFAs) like propionate, acetate, and butyrate, and also secondary bile acids and ubiquitous bacterial fermentation products. SCFAs act as effective inhibitors of histone deacetylases (HDACs) and lysine deacetylase (KDAC) in innate immune cells such as macrophages and DCs. In contrast, when dysbiosis occurs, it can directly or indirectly result in functional impairment of local, locoregional, and systemic immune responses leading to disintegration of epithelial barriers, and subsequently delivery of mucosa-associated microbes and their components into the mesenteric lymph nodes (MLNs) and into the peripheral circulation. In addition, ligand-receptor interactions between natural killer (NK) cells and healthy gut microbiota are critical for modulating the immune system. HMOs, such as 3′-SL, 6′-SL, and 2′-FL as well as certain derivatives thereof, can restore (or reduce) the disequilibrium and thus modulate the immune response. Furthermore, HMOs can inhibit one or more oncomicrobes. Oncomicrobes are biological pathogens that cause cancer and include, for example, human papilloma virus (HPV), Epstein-Barr virus (EBV), hepatitis B and C viruses (HBV and HCV) and H. pylori.

Derivatives of natural HMOs can be chemically modified as compared to the natural HMO. In certain aspects, the derivative of the natural HMO retains at least 50%, at least 60%, at least 70% or more (including, e.g., at least 80%, at least 90%, at least 95%, at least 98%, at least 99% and up to 100%) of the biological functions of a natural HMO. Such biological effects include, but are not limited to, anti-inflammatory effects, anti-bacterial adhesion effects, prebiotic effects and/or or effects in treating or preventing the spaceflight-associated disease or condition and/or the disease or condition associated with a low gravity environment.

HMOs include, but are not limited to, compounds having a structure of Formula I, I(a), II, IIa, or III(b):

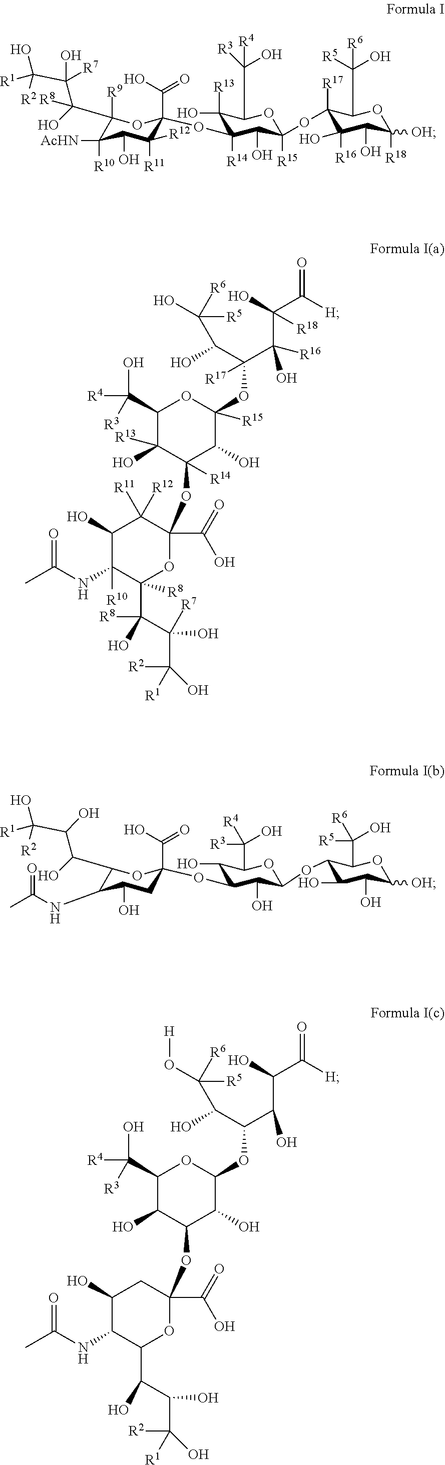

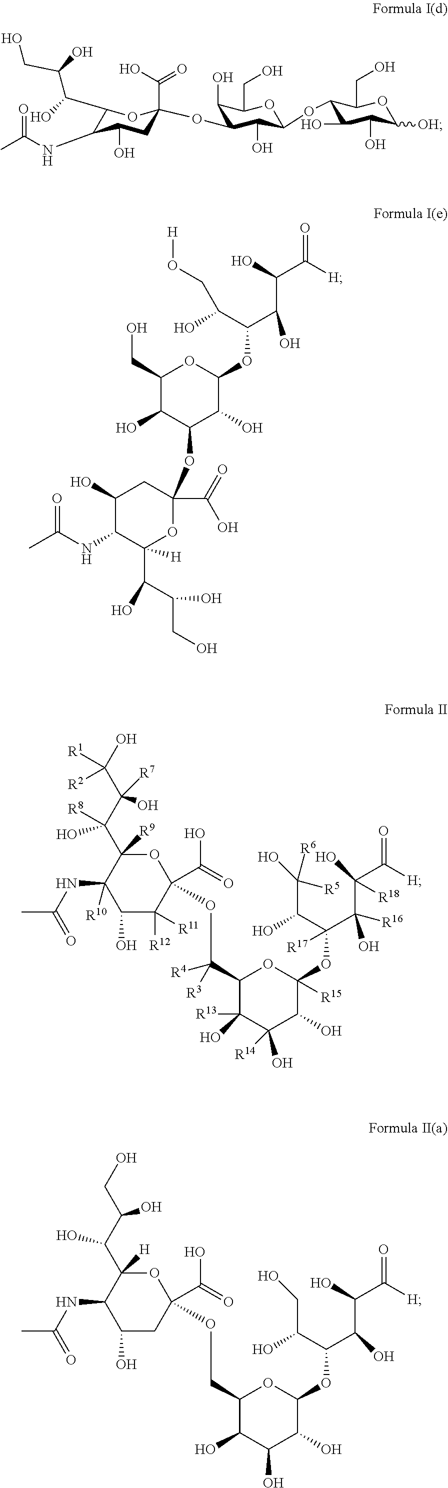

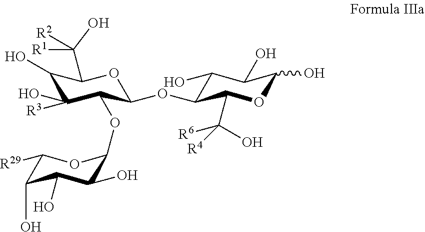

or a pharmaceutically acceptable salt, solvate, or prodrug thereof, wherein:
R¹-R¹⁸ are each independently selected from H, D, a halo, an unsubstituted or substituted (C₁-C₆)alkyl, an unsubstituted or substituted (C₁-C₆)heteroalkyl, an unsubstituted or substituted (C₂-C₆)alkenyl, an unsubstituted or substituted (C₂-C₆)heteroalkenyl, an unsubstituted or substituted (C₃-C₆)alkynyl, an unsubstituted or substituted (C₃-C₆)heteroalkynyl, an unsubstituted or substituted (C₄-C₈)cycloalkyl, an unsubstituted or substituted heterocycle, an unsubstituted or substituted aryl, —ROR′, —RN(R′)₂, —RSSR′, —SH, —RSOR′, —RSO₂R′, —RSO₂H, —RSO₃H, —RC(═S)—R′, —ROH, —RC(═O)R′, —RNO₂, —RSR′, —RCN, —RNC, —RNNR′, —RC(═O)OR′, —ROC(═O)R′, —RC(═O)H, —RC(═O)OH, —RC(═O)N(R′)₂, —RN₃, —ROCN, —RNCO, —RONO₂, —RNO, —ROP(═OXOH)₂, and —RB(OH)₂;
R is absent or a (C₁-C₅)alkyl;
R′ is independently selected from H, D, an unsubstituted or substituted (C₁-C₆) alkyl, an unsubstituted or substituted (C₁-C₆)heteroalkyl, an unsubstituted or substituted (C₂-C₆) alkenyl, an unsubstituted or substituted (C₂-C₆)heteroalkenyl, an unsubstituted or substituted (C₃-C₆)alkynyl, an unsubstituted or substituted(C₃-C₆)heteroalkynyl, an unsubstituted or substituted (C₄-C₈)cycloalkyl, an unsubstituted or substituted heterocycle, and an unsubstituted or substituted aryl; and
R²⁹ is an unsubstituted or substituted (C₁-C₆)alkyl.

In yet additional aspects, the HMO has the Formula IIIb:

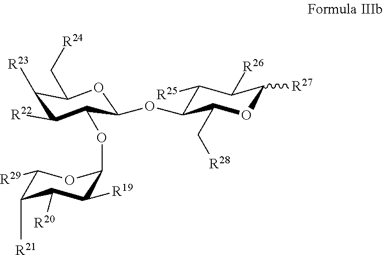

wherein:
one, two or three of R¹⁹-R²⁸ are each independently selected from the group consisting of hydrogen, an unsubstituted or substituted C₁-C₆ alkyl (including, but not limited to, methyl and ethyl) and N(R′)2 (wherein R′ is as defined above), the remainder or R₁₉-R₂₈ are —OH, and R²⁹ is substituted or unsubstituted C₁-C⁶ alkyl; or
one, two or three of R¹⁹-R²⁹ are each independently selected from NHC(O)R″, wherein R″ is unsubstituted or substituted (C₁-C₆) alkyl (including, but not limited to, methyl), the remainder or R₁₉-R₂₈ are —OH, and R²⁹ is substituted or unsubstituted C₁-C⁶ alkyl. In certain aspects, R²⁶ is NHC(O)CH₃ and R¹⁹-R²⁵ and R²⁷-R²⁸ s are —OH, and R²⁹ is methyl.

The term “alkyl” refers to an organic group that is comprised of carbon and hydrogen atoms that contains single covalent bonds between carbons. Typically, an “alkyl” as used in this disclosure, refers to an organic group that contains 1, 2, 3, 4, 5, 6, 7, 8, 9, 10, 11, 12, 13, 14, 15, 16, 17, 18, 19, 20, or 30 carbon atoms, or any range of carbon atoms between or including any two of the foregoing values. Where if there is more than 1 carbon, the carbons may be connected in a linear manner, or alternatively if there are more than 2 carbons then the carbons may also be linked in a branched fashion so that the parent chain contains one or more secondary, tertiary, or quaternary carbons. An alkyl may be substituted or unsubstituted, unless stated otherwise.

The term “alkenyl” refers to an organic group that is comprised of carbon and hydrogen atoms that contains at least one double covalent bond between two carbons. Typically, an “alkenyl” as used in this disclosure, refers to organic group that contains 2, 3, 4, 5, 6, 7, 8, 9, 10, 11, 12, 13, 14, 15, 16, 17, 18, 19, 20, or 30 carbon atoms, or any range of carbon atoms between or including any two of the foregoing values. While a C2-alkenyl can form a double bond to a carbon of a parent chain, an alkenyl group of three or more carbons can contain more than one double bond. In certain instances, the alkenyl group will be conjugated, in other cases an alkenyl group will not be conjugated, and yet other cases the alkenyl group may have stretches of conjugation and stretches of non-conjugation. Additionally, if there is more than 2 carbon, the carbons may be connected in a linear manner, or alternatively if there are more than 3 carbons then the carbons may also be linked in a branched fashion so that the parent chain contains one or more secondary, tertiary, or quaternary carbons. An alkenyl may be substituted or unsubstituted, unless stated otherwise.

The term “alkynyl”, refers to an organic group that is comprised of carbon and hydrogen atoms that contains a triple covalent bond between two carbons. Typically, an “alkynyl” as used in this disclosure, refers to organic group that contains that contains 2, 3, 4, 5, 6, 7, 8, 9, 10, 11, 12, 13, 14, 15, 16, 17, 18,19, 20, or 30 carbon atoms, or any range of carbon atoms between or including any two of the foregoing values. While a C2-alkynyl can form a triple bond to a carbon of a parent chain, an alkynyl group of three or more carbons can contain more than one triple bond.

Where if there is more than 3 carbon, the carbons may be connected in a linear manner, or alternatively if there are more than 4 carbons then the carbons may also be linked in a branched fashion so that the parent chain contains one or more secondary, tertiary, or quaternary carbons. An alkynyl may be substituted or unsubstituted, unless stated otherwise.

The term “aryl”, as used in this disclosure, refers to a conjugated planar ring system with delocalized pi electron clouds that contain only carbon as ring atoms. An “aryl” for the purposes of this disclosure encompass from 1 to 4 aryl rings wherein when the aryl is greater than 1 ring the aryl rings are joined so that they are linked, fused, or a combination thereof. An aryl may be substituted or unsubstituted, or in the case of more than one aryl ring, one or more rings may be unsubstituted, one or more rings may be substituted, or a combination thereof.

The term “cycloalkyl”, as used in this disclosure, refers to an alkyl that contains at least 3 carbon atoms but no more than 12 carbon atoms connected so that it forms a ring. A “cycloalkyl” for the purposes of this disclosure encompasses from 1 to 4 cycloalkyl rings, wherein when the cycloalkyl is greater than 1 ring, then the cycloalkyl rings are joined so that they are linked, fused, or a combination thereof. A cycloalkyl may be substituted or unsubstituted, or in the case of more than one cycloalkyl ring, one or more rings may be unsubstituted, one or more rings may be substituted, or a combination thereof.

The term “hetero-” when used as a prefix, such as, hetero-alkyl, hetero-alkenyl, hetero-alkynyl, or hetero-hydrocarbon, for the purpose of this disclosure refers to the specified hydrocarbon having one or more carbon atoms replaced by non-carbon atoms as part of the parent chain. Examples of such non-carbon atoms include, but are not limited to, N, O, S, Si, Al, B, and P. If there is more than one non-carbon atom in the hetero-based parent chain then this atom may be the same element or may be a combination of different elements, such as N and O. In a particular embodiment, a “hetero”-hydrocarbon (e.g., alkyl, alkenyl, alkynyl) refers to a hydrocarbon that has from 1 to 3 C, N and/or S atoms as part of the parent chain.

The term “heterocycle,” as used herein, refers to ring structures that contain at least 1 noncarbon ring atom. A “heterocycle” for the purposes of this disclosure encompass from 1 to 4 heterocycle rings, wherein when the heterocycle is greater than 1 ring the heterocycle rings are joined so that they are linked, fused, or a combination thereof. A heterocycle may be aromatic or nonaromatic, or in the case of more than one heterocycle ring, one or more rings may be nonaromatic, one or more rings may be aromatic, or a combination thereof. A heterocycle may be substituted or unsubstituted, or in the case of more than one heterocycle ring one or more rings may be unsubstituted, one or more rings may be substituted, or a combination thereof. Typically, the noncarbon ring atom is N, O, S, Si, Al, B, or P. In the case where there is more than one noncarbon ring atom, these noncarbon ring atoms can either be the same element, or combination of different elements, such as N and O.

Examples of heterocycles include, but are not limited to: a monocyclic heterocycle such as, aziridine, oxirane, thiirane, azetidine, oxetane, thietane, pyrrolidine, pyrroline, imidazolidine, pyrazolidine, pyrazoline, dioxolane, sulfolane 2,3-dihydrofuran, 2,5-dihydrofuran tetrahydrofuran, thiophane, piperidine, 1,2,3,6-tetrahydro-pyridine, piperazine, morpholine, thiomorpholine, pyran, thiopyran, 2,3-dihydropyran, tetrahydropyran, 1,4-dihydropyridine, 1,4-dioxane, 1,3-dioxane, dioxane, homopiperidine, 2,3,4,7-tetrahydro-1H-azepine homopiperazine, 1,3-dioxepane, 4,7-dihydro-1,3-dioxepin, and hexamethylene oxide; and polycyclic heterocycles such as, indole, indoline, isoindoline, quinoline, tetrahydroquinoline, isoquinoline, tetrahydroisoquinoline, 1,4-benzodioxan, coumarin, dihydrocoumarin, benzofuran, 2,3-dihydrobenzofuran, isobenzofuran, chromene, chroman, isochroman, xanthene, phenoxathiin, thianthrene, indolizine, isoindole, indazole, purine, phthalazine, naphthyridine, quinoxaline, quinazoline, cinnoline, pteridine, phenanthridine, perimidine, phenanthroline, phenazine, phenothiazine, phenoxazine, 1,2-benzisoxazole, benzothiophene, benzoxazole, benzthiazole, benzimidazole, benztriazole, thioxanthine, carbazole, carboline, acridine, pyrolizidine, and quinolizidine. In addition to the polycyclic heterocycles described above, heterocycle includes polycyclic heterocycles wherein the ring fusion between two or more rings includes more than one bond common to both rings and more than two atoms common to both rings. Examples of such bridged heterocycles include quinuclidine, diazabicyclo[2.2.1]heptane and 7-oxabicyclo[2.2.1]heptane.

The terms “heterocyclic group”, “heterocyclic moiety”, “heterocyclic”, or “heterocyclo” used alone or as a suffix or prefix, refers to a heterocycle that has had one or more hydrogens removed there from.

The term “hydrocarbons” refers to groups of atoms that contain only carbon and hydrogen. Examples of hydrocarbons that can be used in this disclosure include, but are not limited to, alkanes, alkenes, alkynes, arenes, and benzyls.

The term “optionally substituted” means independent replacement of one or more hydrogen atoms with a substituent. The term “optionally substituted” also refers to a functional group, typically a hydrocarbon or heterocycle, where one or more hydrogen atoms may be replaced with a substituent. Accordingly, “optionally substituted” refers to a functional group that is substituted, in that one or more hydrogen atoms are replaced with a substituent, or unsubstituted, in that the hydrogen atoms are not replaced with a substituent.

For example, an optionally substituted hydrocarbon group refers to an unsubstituted hydrocarbon group or a substituted hydrocarbon group.

As described herein, the subject is administered a composition comprising one or more human milk oligosaccharides. The composition can comprise 10% or more, 20% or more, 30% or more, 40% or more, or 50% or more by mass one or more human milk oligosaccharide. In certain aspects, the composition is not human milk. In additional aspects, the composition is not derived from human milk.

In certain aspects, the one or more human milk oligosaccharides are selected from lacto-N-tetraose (LNT), lacto-N-neotetraose (LNnT), lacto-N-hexaose (LNH), lacto-N-neohexaose (LNnH), 2′fucosyllactose (2′FL), 3′fucosyllacose (3′FL), lacto-difucotetraose (LDFT), lacto-N-fucopenaose II/III (LNFP II/III), lactose-N-fucopentaose I (LNFP I), lacto-N-difuco-hexaose I (LNDFH I), lacto-N-difuco-hexaose II (LNDFH II), difucosyl-para-lacto-N-neohexaose (DFpLNnH), difucosyllacto-N-hexaose c (DFLNH c), 3′ sialyllactose (3′SL), 6′ sialyllactose (6′SL), LS-tetrasaccharide a (LSTa), LS-tetrasaccharide b (LST b), LS-tetrasaccharide c (LST c), 3′-sialyl-N-acetyllactosamine (3′SLN), 6′-sialyl-N-acetyllactosamine (6′SLN), or disialyllacto-N-tetraose (DSLNT), or a combination of any of thereof. In yet other aspects, the one or more human milk oligosaccharides are selected from 2′FL, 3′FL, 3′SL, 6′SL, LNT, or LNnT, or a combination of any of thereof.

The composition administered to the subject can comprise one HMO or can comprise a mixture of two, three, four, five or more HMOs. In certain aspects, the composition comprises one HMO and the HMO is selected from the group consisting of 2′FL, 3′FL, 3′SL, 6′SL, LNT, or LNnT.

In further aspects, the composition comprises a mixture of 2′FL and at least one other HMO. In certain aspects, the composition comprises 2′FL and LNT; 2′FL and LNnT; 2′FL, 3′FL, 3′SL, 6′SL and LNT. The composition comprising 2′FL and LNT includes a 4:1 mixture of 2′FL and LNT; such a composition is GRAS (generally regarded as safe) and is available from Glycom, Lyngby, Denmark. A composition comprising 2′FL, 3′FL, 3′SL, 6′SL and LNT is sold by Jennewein Biotechnologie and is GRAS.

In further aspects, the composition comprises a mixture of one neutral core and one neutral fucosylated human milk oligosaccharide. In additional embodiments, the composition comprises a mixture of one neutral and one acidic human milk oligosaccharide. In certain aspects, the human milk oligosaccharide used in the compositions and methods described herein are selected from 2′-FL, 3′-SL and 6′-SL, as well as a combination of any of thereof.

Human milk oligosaccharides, including 2′-fucosyllactose, 3′ sialyllactose, and 6′ sialyllactose, can be readily prepared with well-established synthetic biology methods.

In some embodiments, the human milk oligosaccharide can efficiently promote antigen uptake and migration of dendritic cells to lymph nodes where tumor-specific antigens are presented by dendritic cells to prime immune effector cells. Exposure of dendritic cells to human milk oligosaccharides can induce maturation of dendritic cells and significantly increase their capacity to prime T-cells. In some embodiments, human milk oligosaccharides can mediate tumor size reduction through immune modulation of the tumor microenvironment to promote anti-tumor immune enhancing effects. In some embodiments, substantial therapeutic synergies can be achieved when combining the human milk oligosaccharide with an immune checkpoint inhibitor as described herein.

Immune checkpoint inhibitors include, but are not limited, inhibitors of CTLA4 (cytotoxic T lymphocyte antigen-4), PD-1 (programmed cell death protein 1), PD-L1 (programmed cell death ligand 1), PD-L2 (programmed cell death ligand 2), PD-L3 (programmed cell death ligand 3), PD-L4 (programmed cell death ligand 4), LAG-3 (lymphocyte activation gene-3), and TIM-3 (T cell immunoglobulin and mucin protein-3). In some embodiments, the immune checkpoint inhibitor is a binding ligand of PD-1. In some embodiments, the immune checkpoint inhibitor is a binding ligand of CTLA-4. The terms “immune checkpoint inhibitor” and “checkpoint inhibitor” are used interchangeably herein.

PD-1 is a key immune checkpoint receptor expressed by activated T and B cells and mediates immunosuppression. PD-1 is a member of the CD28 family of receptors, which includes CD28, CTLA-4, ICOS, PD-1, and BTLA. The term “PD-1” as used herein includes human PD-1 (hPD-1), variants, isoforms, and species homologs of hPD-1, and analogs having at least one common epitope with hPD-1.

Various cell surface glycoprotein ligands for PD-1 have been identified, including PD-L1, PD-L2, PD-L3, and PD-L4, that are expressed on antigen-presenting cells as well as many human cancers and have been shown to downregulate T cell activation and cytokine secretion upon binding to PD-1. The term “PD-L1” as used herein includes human PD-L1 (hPD-L1), variants, isoforms, and species homologs of hPD-L1, and analogs having at least one common epitope with hPD-L1. The term “PD-L2” as used herein includes human PD-L2 (hPD-L2), variants, isoforms, and species homologs of hPD-L2, and analogs having at least one common epitope with hPD-L2. The term “PD-L3” as used herein includes human PD-L3 (hPD-L3), variants, isoforms, and species homologs of hPD-L3, and analogs having at least one common epitope with hPD-L3. The term “PD-L4” as used herein includes human PD-L4 (hPD-L4), variants, isoforms, and species homologs of hPD-L4, and analogs having at least one common epitope with hPD-L4. CTLA-4 (cytotoxic T-lymphocyte-associated protein 4) is a protein receptor that, functioning as an immune checkpoint, downregulates the immune system. CTLA4 is found on the surface of T cells, is also a member of the immunoglobulin (Ig) superfamily; CTLA-4 comprises a single extracellular Ig domain. CTLA-4 transcripts have been found in T cell populations having cytotoxic activity, suggesting that CTLA-4 might function in the cytolytic response.

In certain aspects, the immune checkpoint inhibitor is selected from the group consisting of ipilimumab (YERVOY; Bristol-Myers Squibb), nivolumab (Bristol-Meyers Squibb), pembrolizumab (Merck), pidilizumab (Curetch), arelumab (Merck Serono), tremelimumab (Pfizer), atezolizumab, AMP-224 (GlaxoSmithKline/Amplimmune), MPDL3280A (Roche), MDX-1105 (Medarex, Inc/Bristol-Meyer Squibb), MDX-1106, MEDI-4736, IMP321, INCB024360, NLG-919, indoximod, AUNP 12, galiximab (Biogen Idec), avelumab (EMD Serono), varlilumab (CeliDex Therapeutics), mogamulizumab (Kyowa Hakko Kirin), CP-870,893, MEDI-6469 (MedImmune), IPH2101 (Innate Pharma/Bristol-Meyers Squibb), urelumab (Bristol-Meyers Squibb), lirilumab (Bristol-Meyers Squibb), BMS-986016 (Bristol-Meyers Squibb), MGA271, IMP321, BMS-936559, MSB0010718C, anti-OX40, MK-3475, CT-011, BY55, AMP224, and BGB-A317.

In certain aspects, the composition comprises a PD-1 inhibitor, for example, an anti-PD-1 antibody. In yet additional aspects, the composition comprises a PD-L1 inhibitor, for example, an anti-PD-L1 antibody. In yet further aspects, the composition comprises a CTLA-4 inhibitor, for example, an anti-CTLA-4 antibody. The composition can comprise 2′fucosyllactose, 3′ sialyllactose, and/or 6′ sialyllactose and can further comprise an immune checkpoint inhibitor selected from the group consisting of a PD-1 inhibitor, a PD-L1 inhibitor, and a CTLA-4 inhibitor, or a combination thereof. In another example, the composition can comprise 2′fucosyllactose, 3′ sialyllactose, and/or 6′ sialyllactose and can further comprise an immune checkpoint inhibitor selected from the group consisting of an anti-PD-1 antibody, an anti-PD-L1 antibody, and an anti-CTLA-4 antibody, or a combination thereof. The composition can comprise more than one checkpoint inhibitor. For example, the composition can comprise at least a first checkpoint inhibitor and a second checkpoint inhibitor, wherein the first and second checkpoint inhibitors are different, and wherein the first and second checkpoint inhibitors are each independently an inhibitor of PD-1, PD-L1, PD-L2, PD-L3, PD-L4, CTLA-4, LAG3, B7-H3, B7-H4, KIR or TIM3. In yet further aspects, the first immune checkpoint inhibitor is selected from the group consisting of a PD-1 inhibitor, a PD-L1 inhibitor and PD-L2 inhibitor and the second checkpoint inhibitor is CTLA-4.

In some embodiments, the composition comprises at least 9% (e.g., 10, 15, 20, 25, 30, 35, 40, 45, 50, 55, 60, 65, 70, 75, 80, 85, 90, 95, or 100%; or any value between any of the foregoing) 3′SL, 6′SL, or 2′FL, or a combination thereof, of the total oligosaccharides in the composition. In certain aspects, the pharmaceutical composition is formulated as a tablet or a capsule; for example, wherein the immune checkpoint inhibitor is a small molecule. In yet additional aspects, the pharmaceutical composition is formulated as an injectable composition.

In certain aspects, the pharmaceutical composition comprises 3′SL, 6′SL and/or 2′-FL, or a combination thereof, and optionally a pharmaceutically acceptable carrier or excipient. In certain embodiments, the composition comprises or consists of 3′SL and 6′SL or a combination of 3′SL and 6′SL, and optionally a pharmaceutically acceptable carrier or excipient.

The invention additionally encompasses a method of treating cancer comprising administering to a patient the pharmaceutical composition described herein. The invention additionally encompasses a method of treating cancer in a patient in need thereof comprising co-administering to the patient an effective amount of a human milk oligosaccharide and an effective amount of an immune checkpoint inhibitor.

The patient or subject to be treated can be a patient or subject suffering from cancer. In preferred aspects, the patient is a human patient that is 5 years of age or older (e.g., 6, 7, 8, 9, 10, 11, 12 years of age or older). In yet other aspects, the patient is less than 5 years of age. In other aspects, the patient is 18 years of age or older.

In certain aspects, the invention is directed to a method of treating cancer wherein the oligosaccharide is administered orally, for example. In certain aspects, the immune checkpoint is administered by injection including, for example, intravenous injection, intra-tumoral injection (e.g., local injection into the tumor or melanoma), and peri-tumoral injection. The human milk oligosaccharide can be administered to the subject before, during or after beginning immune checkpoint inhibitor treatment. In certain aspects, the human milk oligosaccharide is orally administered to the subject before, during or after administration of the immune checkpoint inhibitor.

The oligosaccharide can be administered in an amount from about 1 g to about 20 g per day or per dose. The oligosaccharide can be administered at various intervals, for example, once a day, twice a day, three times a day, once a week, twice a week, at the same time or at substantially the same time as the immune checkpoint inhibitor, or as needed.

In certain aspects, the method comprises administration of an effective amount of a PD-1 inhibitor, for example, an anti-PD-1 antibody and more specifically an anti-PD-1 monoclonal antibody (mAb). An exemplary anti-PD-1 mAb is nivolumab. In yet additional aspects, the method comprises administration of an effective amount of a PD-L1 inhibitor, for example, an anti-PD-L1 antibody, and more specifically an anti-PD-L1 mAb. Exemplary anti-PD-L1 mAbs are atezolizumab, avelumab, and durvalumab. In yet further aspects, the method comprises administration of an effective amount of a CTLA-4 inhibitor, for example, an anti-CTLA-4 antibody, and more specifically an anti-CTLA-4 mAb. An exemplary anti-CTLA4 mAb is ipilimumab. The method can comprise administration of an effective amount of 2′fucosyllactose, 3′ sialyllactose, and/or 6′ sialyllactose and can further comprise administration of an effective amount of an immune checkpoint inhibitor selected from the group consisting of a PD-1 inhibitor, a PD-L1 inhibitor, and a CTLA-4 inhibitor, or a combination thereof. In another example, the method comprises administration of an effective amount of 2′fucosyllactose, 3′ sialyllactose, and/or 6′ sialyllactose and can further comprise administration of an effective amount of an immune checkpoint inhibitor selected from the group consisting of an anti-PD-1 antibody, an anti-PD-L1 antibody, and an anti-CTLA-4 antibody, or a combination thereof. The method can additionally comprise administration of more than one checkpoint inhibitor. For example, the method can comprise administration of an effective amount of a first checkpoint inhibitor and a second checkpoint inhibitor, wherein the first and second checkpoint inhibitors are different, and wherein the first and second checkpoint inhibitors are each independently an inhibitor of PD-1, PD-L1, PD-L2, PD-L3, PD-L4, CTLA-4, LAG3, B7-H3, B7-H4, KIR or TIM3. In yet further aspects, the first immune checkpoint inhibitor is selected from the group consisting of a PD-1 inhibitor, a PD-L1 inhibitor and PD-L2 inhibitor and the second checkpoint inhibitor is CTLA-4.

The dose of the immune checkpoint inhibitor can be appropriately adjusted depending on factors such as the condition of the patient, the type and severity of the disease of the patient. For example, a suitable dose of an immune checkpoint inhibitor may be at least 0.1 mg/kg. For example, the dose of the immune checkpoint inhibitor that is administered per kg body weight of the patient may be at least 0.5 mg/kg. As another example, the dose of the immune checkpoint inhibitor that is administered per kg body weight of the patient may be at least 1 mg/kg. In another example, the immune checkpoint inhibitor that is administered per kg body weight may be at least 2 mg/kg, such as at least 3 mg/kg, and even at least 10 mg/kg, such as at least 15 mg/kg. Generally, the dose of the immune checkpoint inhibitor that is administered per kg body weight of the patient will not exceed 20 mg/kg, such as a dose that does not exceed 15 mg/kg, and even that does not exceed 10 mg/kg. For example, the dose of the immune checkpoint inhibitor that is administered per kg body weight of the patient may be in the range of from 0.1 to 15 mg/kg. As another example, the dose of the immune checkpoint inhibitor that is administered per kg body weight may be in a range of from 2 mg/kg to 15 mg/kg. As yet another example, the dose of the immune checkpoint inhibitor that is administered per kg body weight may be in a range of from 3 mg/kg to 10 mg/kg.

The dosing schedule of the immune checkpoint inhibitor can similarly be selected according to the intended treatment and the particular immune checkpoint inhibitor being provided. For example, in one embodiment, a suitable dosing schedule in one embodiment can comprise dosing a patient once every 2 or 3 weeks, for a total of 4 doses (9 weeks of treatment total). That is, in some embodiments, treatment may involve a course of therapy that lasts at least 9 weeks and even 10 weeks, but in some embodiments may not extend past 16 weeks. In particular, the package insert for Yervoy (ipilimumab) indicates that a dose of 3 mg/kg should be given every 3 weeks for 4 doses, as given by IV over the course of 90 minutes. Dosage regimens for Opdivo (nivolumab) and Keytruda (pembrolizumab) similarly indicate dosing once every 2 or 3 weeks.

The method can further comprise administration of an effective amount of an additional chemotherapeutic agent.

The cancer to be treated can express a binding ligand of a checkpoint protein expressed by an immune cell. For example, in certain aspects, the cancer comprises cancer cells that express a binding ligand of PD-1 (e.g., PD-L1 or PD-L2) or a binding ligand of CTLA-4 and the immune checkpoint inhibitor is one that inhibits the binding interaction between the checkpoint protein expressed by the immune cell and the binding ligand expressed by the cancer cell. In certain aspects, the cancer comprises cells that express a binding ligand of PD-1. Exemplary binding ligands of PD-1 are PD-L1 and PD-L2. In other aspects, the cancer comprises cells that express a binding ligand of CTLA-4. The binding ligand of CTLA-4 can be B7.1 or B7.2. In yet other aspects, the cancer does not comprise cells expressing PD-1, PD-L1, or PD-L2.

In some examples, the patient is suffering from a cancer selected from the group consisting of breast cancer, colon cancer, rectal cancer, lung cancer, prostate cancer, melanoma, leukemia, ovarian cancer, gastric cancer, renal cell carcinoma, liver cancer, pancreatic cancer, lymphoma and myeloma. Additional examples of cancer that can be treated are renal cell carcinoma, malignant melanoma, non-small cell lung cancer (NSCLC), ovarian cancer, Hodgkin's lymphoma or squamous cell carcinoma. The cancer to be treated can also be a solid tumor or a hematologic cancer. The patient can be treated by administering one or more oligosaccharides selected from the group consisting of 2′fucosyllactose, 3′ sialyllactose and 6′ sialyllactose and an effective amount of an immune checkpoint inhibitor.

In additional embodiments, the invention is a method of treating melanoma in a patient in need thereof comprising administering to the patient an effective an oligosaccharide selected from the group consisting of 2′fucosyllactose, 3′ sialyllactose and 6′ sialyllactose and an effective amount of an immune checkpoint inhibitor selected from the group consisting of ipilimumab.

In yet additional embodiments, the invention is a method of treating bladder cancer, classical Hodgkin lymphoma, colorectal cancer, hepatocellular cancer, non-small-cell lung cancer, kidney cancer, squamous cell carcinoma of the head and neck and urothelial cancer in a patient in need thereof comprising administering to the patient an effective an oligosaccharide selected from the group consisting of 2′fucosyllactose, 3′ sialyllactose and 6′ sialyllactose and an effective amount of an immune checkpoint inhibitor selected from the group consisting of nivolumab.

In additional aspects, the invention is a method of treating melanoma, lung cancer, head and neck cancer, Hodgkin lymphoma, stomach cancer (e.g., advanced gastric cancer), microsatellite instability-high cancer, or advanced urothelial bladder cancer in a patient in need thereof comprising administering to the patient an effective amount of an oligosaccharide selected from the group consisting of 2′fucosyllactose, 3′ sialyllactose and 6′ sialyllactose and an effective amount of pembrolizumab.

The invention includes a method of treating diffuse large B-cell lymphoma, relapsed follicular lymphoma or glioma in a patient in need thereof comprising administering to the patient an effective amount of an oligosaccharide selected from the group consisting of 2′fucosyllactose, 3′ sialyllactose and 6′ sialyllactose and an effective amount of pidilizumab.

In further aspects, the invention is directed to a method of treating melanoma or Hodgkin's lymphoma comprising administering to the patient an effective amount of an oligosaccharide selected from the group consisting of 2′fucosyllactose, 3′ sialyllactose and 6′ sialyllactose and an effective amount of dacarbazine.

In yet further embodiments, the invention is a method of treating urothelial carcinoma, non-small cell lung cancer, triple-negative breast cancer, small cell lung cancer, or hepatocellular carcinoma comprising administering to the patient an effective amount of an oligosaccharide selected from the group consisting of 2′fucosyllactose, 3′ sialyllactose and 6′ sialyllactose and an effective amount of atezolizumab.

The invention also includes a method for treating urothelial carcinoma or lung cancer (including, for example, unresectable Stage III non-small cell lung cancer (NSCLC) and extensive-stage small cell lung cancer) comprising administering to the patient suffering therefrom with an effective amount of an oligosaccharide selected from the group consisting of 2′fucosyllactose, 3′ sialyllactose and 6′ sialyllactose and an effective amount of durvalumab.

The invention also encompasses a method of treating Merkel cell carcinoma or urothelial cancer comprising administering to the patient suffering therefrom with an effective amount of an oligosaccharide selected from the group consisting of 2′fucosyllactose, 3′ sialyllactose and 6′ sialyllactose and an effective amount of avelumab.

In certain aspects, the HMO is administered orally. In certain aspects, the HMO is administered in a composition other than mammalian breast milk, for example, the composition is other than human breast milk. The compositions and methods described herein can be used to prevent or inhibit progression of the cancer or tumor, improve the efficacy of the immune checkpoint inhibitor, reduce the effective dose of the immune checkpoint inhibitor, and/or reduce the toxicity of the immune checkpoint inhibitor. The compositions and methods can also be used to prevent or delay the incidence of cancer in a subject, for example, a subject at risk of developing cancer.

In further aspects, the methods additionally provide an improvement in the subject's microbiota (gut and/or oral) composition. Improvement in, or avoidance of, gastrointestinal symptoms, such as constipation, diarrhea, stool consistency, stool smell, flatulence and abdominal pain is desirable, such as, for example, at weeks 8 and 16 of the beginning of treatment.

Oral administration of the oligosaccharides of the disclosure provide for systemic circulation of the oligosaccharides both in infants and adults. Unlike other drug products approved by the FDA, the oligosaccharides described herein can not only be administered to treat a disease or disorder in an adult subject, but can also be administered to pregnant females, infants, and subjects who have impaired organ function (e.g., liver disfunction, kidney failure). Due to the oligosaccharides of the disclosure having little to no adverse effects in humans, this form of therapy could be used as a preventive, as a first line therapy option, or as an adjunct to existing therapies that would be well tolerated by patients of either sex.

In a further embodiment, said oligosaccharide is substantially a single enantiomer, a mixture of about 90% or more by weight of the (−)-enantiomer and about 10% or less by weight of the (+)-enantiomer, a mixture of about 90% or more by weight of the (+)-enantiomer and about 10% or less by weight of the (−)-enantiomer, substantially an individual diastereomer, or a mixture of about 90% or more by weight of an individual diastereomer and about 10% or less by weight of any other diastereomer.

The oligosaccharides disclosed herein may be enantiomerically pure, such as a single enantiomer or a single diastereomer, or be stereoisomeric mixtures, such as a mixture of enantiomers, a racemic mixture, or a diastereomeric mixture. As such, one of skill in the art will recognize that administration of an oligosaccharide in its (R) form is equivalent, for oligosaccharides that undergo epimerization in vivo, to administration of the oligosaccharide in its (S) form. Conventional techniques for the preparation/isolation of individual enantiomers include chiral synthesis from a suitable optically pure precursor or resolution of the racemate using, for example, chiral chromatography, recrystallization, resolution, diastereomeric salt formation, or derivatization into diastereomeric adducts followed by separation.

When the oligosaccharide disclosed herein contains an acidic or basic moiety, it may also be disclosed as a pharmaceutically acceptable salt (See, Berge et al., J. Pharm. Sci. 1977, 66, 1-19; and “Handbook of Pharmaceutical Salts, Properties, and Use,” Stah and Wermuth, Ed.; Wiley-VCH and VHCA, Zurich, 2002).

Suitable acids for use in the preparation of pharmaceutically acceptable salts include, but are not limited to, acetic acid, 2,2-dichloroacetic acid, acylated amino acids, adipic acid, alginic acid, ascorbic acid, L-aspartic acid, benzenesulfonic acid, benzoic acid, 4-acetamidobenzoic acid, boric acid, (+)-camphoric acid, camphorsulfonic acid, (+)-(1S)-camphor-10-sulfonic acid, capric acid, caproic acid, caprylic acid, cinnamic acid, citric acid, cyclamic acid, cyclohexanesulfamic acid, dodecylsulfuric acid, ethane-1,2-disulfonic acid, ethanesulfonic acid, 2-hydroxy-ethanesulfonic acid, formic acid, fumaric acid, galactaric acid, gentisic acid, glucoheptonic acid, D-gluconic acid, D-glucuronic acid, L-glutamic acid, a-oxo-glutaric acid, glycolic acid, hippuric acid, hydrobromic acid, hydrochloric acid, hydroiodic acid, (+)-L-lactic acid, (±)-DL-lactic acid, lactobionic acid, lauric acid, maleic acid, (−)-L-malic acid, malonic acid, (±)-DL-mandelic acid, methanesulfonic acid, naphthalene-2-sulfonic acid, naphthalene-1,5-disulfonic acid, 1-hydroxy-2-naphthoic acid, nicotinic acid, nitric acid, oleic acid, orotic acid, oxalic acid, palmitic acid, pamoic acid, perchloric acid, phosphoric acid, L-pyroglutamic acid, saccharic acid, salicylic acid, 4-amino-salicylic acid, sebacic acid, stearic acid, succinic acid, sulfuric acid, tannic acid, (+)-L-tartaric acid, thiocyanic acid, p-toluenesulfonic acid, undecylenic acid, and valeric acid.

Suitable bases for use in the preparation of pharmaceutically acceptable salts, including, but not limited to, inorganic bases, such as magnesium hydroxide, calcium hydroxide, potassium hydroxide, zinc hydroxide, or sodium hydroxide; and organic bases, such as primary, secondary, tertiary, and quaternary, aliphatic and aromatic amines, including L-arginine, benethamine, benzathine, choline, deanol, diethanolamine, diethylamine, dimethylamine, dipropylamine, diisopropylamine, 2-(diethylamino)-ethanol, ethanolamine, ethylamine, ethylenediamine, isopropylamine, N-methyl-glucamine, hydrabamine, 1H-imidazole, L-lysine, morpholine, 4-(2-hydroxyethyl)-morpholine, methylamine, piperidine, piperazine, propylamine, pyrrolidine, 1-(2-hydroxyethyl)-pyrrolidine, pyridine, quinuclidine, quinoline, isoquinoline, secondary amines, triethanolamine, trimethylamine, triethylamine, N-methyl-D-glucamine, 2-amino-2-(hydroxymethyl)-1,3-propanediol, and tromethamine.

The oligosaccharide as disclosed herein may also be designed as a prodrug, which is a functional derivative of the oligosaccharide as disclosed herein and is readily convertible into the parent oligosaccharide in vivo. Prodrugs are often useful because, in some situations, they may be easier to administer than the parent oligosaccharide. They may, for instance, be bioavailable by oral administration whereas the parent oligosaccharide is not.

The prodrug may also have enhanced solubility in pharmaceutical compositions over the parent oligosaccharide. A prodrug may be converted into the parent drug by various mechanisms, including enzymatic processes and metabolic hydrolysis. See Harper, Progress in Drug Research 1962, 4, 221-294; Morozowich et al. in “Design of Biopharmaceutical Properties through Prodrugs and Analogs,” Roche Ed., APHA Acad. Pharm. Sci. 1977; “Bioreversible Carriers in Drug in Drug Design, Theory and Application,” Roche Ed., APHA Acad. Pharm. Sci. 1987; “Design of Prodrugs,” Bundgaard, Elsevier, 1985; Wang et al., Curr. Pharm. Design 1999, 5, 265-287; Pauletti et al., Adv. Drug. Delivery Rev. 1997, 27, 235-256; Mizen et al., Pharm. Biotech. 1998, 11, 345-365; Gaignault et al., Pract. Med. Chem. 1996, 671-696; Asgharnejad in “Transport Processes in Pharmaceutical Systems,” Amidon et al., Ed., Marcell Dekker, 185-218, 2000; Balant et al., Eur. J. Drug Metab. Pharmacokinet. 1990, 15, 143-53; Balimane and Sinko, Adv. Drug Delivery Rev. 1999, 39, 183-209; Browne, Clin. Neuropharmacol. 1997, 20, 1-12; Bundgaard, Arch. Pharm. Chem. 1979, 86, 1-39; Bundgaard, Controlled Drug Delivery 1987, 17, 179-96; Bundgaard, Adv. Drug Delivery Rev. 1992, 8, 1-38; Fleisher et al., Adv. Drug Delivery Rev. 1996, 19, 115-130; Fleisher et al., Methods Enzymol. 1985, 112, 360-381; Farquhar et al., J. Pharm. Sci. 1983, 72, 324-325; Freeman et al., J. Chem. Soc., Chem. Commun. 1991, 875-877; Friis and Bundgaard, Eur. J. Pharm. Sci. 1996, 4, 49-59; Gangwar et al., Des. Biopharm. Prop. Prodrugs Analogs, 1977, 409-421; Nathwani and Wood, Drugs 1993, 45, 866-94; Sinhababu and Thakker, Adv. Drug Delivery Rev. 1996, 19, 241-273; Stella et al., Drugs 1985, 29, 455-73; Tan et al., Adv. Drug Delivery Rev. 1999, 39, 117-151; Taylor, Adv. Drug Delivery Rev. 1996, 19, 131-148; Valentino and Borchardt, Drug Discovery Today 1997, 2, 148-155; Wiebe and Knaus, Adv. Drug Delivery Rev. 1999, 39, 63-80; Waller et al., Br. J. Clin. Pharmac. 1989, 28, 497-507.

The oligosaccharide may be produced by biotechnological means using enzyme-based fermentation technology (recombinant or natural enzymes) or microbial fermentation technology. In the latter case, microbes may either express their natural enzymes and substrates or may be engineered to produce respective substrates and enzymes. Single microbial cultures and/or mixed cultures may be used. Alternatively, the oligosaccharides may be produced by chemical synthesis from lactose and other substrates.

Biotechnological approaches have made it possible for the large scale, cost-efficient production of target oligosaccharides.

Precisely, the oligosaccharides disclosed herein can be produced in high yields in aqueous media by fermentation of genetically modified bacteria, yeasts or other microorganisms. See, for example, WO200104341; WO2007101862, WO2010070104; WO2010142305; WO2012112777; Priem et al., Glycobiology 12:235 (2002); Drouillard et al., Angew. Chem. Int. Ed. 45:1778 (2006); Han et al., Biotechnol. Adv. 30:1268 (2012); Lee et al., Microb. Cell Fact. 11:48 (2012); Baumgartner et al., Microb. Cell Fact. 12:40 (2013); and WO 2014135167A1. Alternatively, the oligosaccharides of the disclosure can be synthesized based upon methods described in WO2011100980A1; WO2012007588A1; WO2012127410A1; WO2012155916A1; WO2013044928A1; and U.S. Pat. No. 9,102,966B2. 2′-FL can be made as described in WO 2010/115934 and WO 2010/115935, 3-FL can be made as described in WO 2013/139344. Fucosylated oligosaccharides can be made as described in WO 2012/127410. With regard to biotechnological methods, WO 2001/04341 and WO 2007/101862 describe how to make oligosaccharides optionally substituted by fucose using genetically modified E. coli. The oligosaccharides disclosed herein can be produced in high yields in aqueous media by fermentation of genetically modified bacteria, yeasts or other microorganisms. See, for example, WO200104341; WO2007101862, WO2010070104; WO2010142305; WO2012112777; Priem et al., Glycobiology 12:235 (2002); Drouillard et al., Angew. Chem. Int. Ed. 45:1778 (2006); Han et al., Biotechnol. Adv. 30:1268 (2012); Lee et al., Microb. Cell Fact. 11:48 (2012); Baumgartner et al., Microb. Cell Fact. 12:40 (2013); and WO 2014135167A1.

Alternatively, the oligosaccharides of the disclosure can be synthesized based upon methods described in WO2011100980A1; WO2012007588A1; WO2012127410A1; WO2012155916A1; WO2013044928A1; and U.S. Pat. No. 9,102,966B2. 2′-FL can be made as described in WO 2010/115934 and WO 2010/115935, 3-FL can be made as described in WO 2013/139344, 6′-SL and salts thereof can be made as described in WO 2010/100979, sialylated oligosaccharides can be made as described in WO 2012/113404 and mixtures of human milk oligosaccharides can be made as described in WO 2012/113405. As examples of enzymatic production, sialylated oligosaccharides can be made as described in WO 2012/007588, fucosylated oligosaccharides can be made as described in WO 2012/127410. With regard to biotechnological methods, WO 2001/04341 and WO 2007/101862 describe how to make oligosaccharides optionally substituted by fucose or sialic acid using genetically modified E. coli.

In a certain embodiment, the composition descried herein can further comprise one or more food grade agents. Examples of food grade agents that can be used with the oligosaccharides disclosed herein, include, but are not limited to, milk (e.g., cow's milk, almond milk, soy milk), yogurt, maltodextrin, milk protein concentrate, Sucromalt, glycerine, cocoa powder, soy protein isolate, fructose, vegetable or animal oils (e.g., high oleic safflower oil, soy oil, canola oil), plant sterol esters, HMSs/HMOs, soy lecithin, carrageenan, taurine, L-carnitine, vitamins and/or minerals (e.g., sodium ascorbate, potassium citrate, sodium phosphate, calcium citrate, choline chloride, potassium chloride, sodium citrate, magnesium oxide, alpha-tocopheryl acetate, zinc sulfate, ferrous sulfate, niacinamide, calcium pantothenate, vitamin A palmitate, citric acid, manganese sulfate, pyridoxine hydrochloride, vitamin D3, copper sulfate, thiamine mononitrate, riboflavin, beta carotene, folic acid, biotin, potassium iodide, chromium chloride, sodium selenate, sodium molybdate, phytonadione, vitamin B12, magnesium chloride, calciumphosphate).

Disclosed herein are pharmaceutical compositions comprising one or more oligosaccharides of the disclosure, or a pharmaceutically acceptable salt, solvate, or prodrug thereof, as an active ingredient, combined with a pharmaceutically acceptable vehicle, carrier, diluent, or excipient, or a mixture thereof; in combination with one or more pharmaceutically acceptable excipients or carriers.

Disclosed herein are pharmaceutical compositions in modified release dosage forms, which comprise one or more oligosaccharides of the disclosure, or a pharmaceutically acceptable salt, solvate, or prodrug thereof; and one or more release controlling excipients or carriers as described herein. Suitable modified release dosage vehicles include, but are not limited to, hydrophilic or hydrophobic matrix devices, water-soluble separating layer coatings, enteric coatings, osmotic devices, multiparticulate devices, and combinations thereof.

The pharmaceutical compositions may also comprise non-release controlling excipients or carriers.

Further disclosed herein are pharmaceutical compositions in enteric coated dosage forms, which comprise one or more oligosaccharides (e.g., 3′SL and/or 6′SL and/or 2′FL) as disclosed herein, or a pharmaceutically acceptable salt, solvate, or prodrug thereof; and one or more release controlling excipients or carriers for use in an enteric coated dosage form.

The pharmaceutical compositions may also comprise non-release controlling excipients or carriers.

Further disclosed herein are pharmaceutical compositions in effervescent dosage forms, which comprise one or more oligosaccharides (e.g., 3′SL and/or 6′SL and/or 2′FL) as disclosed herein in substantially pure form (e.g., lacking other oligosaccharides found in milk), or a pharmaceutically acceptable salt, solvate, or prodrug thereof; and one or more release controlling excipients or carriers for use in an effervescent dosage form. The pharmaceutical compositions may also comprise non-release controlling excipients or carriers.

Additionally, disclosed are pharmaceutical compositions in a dosage form that has an instant releasing component and at least one delayed releasing component, and is capable of giving a discontinuous release of one or more oligosaccharides (e.g., 3′SL and/or 6′SL and/or 2′FL disclosed herein in the form of at least two consecutive pulses separated in time (e.g., separated in time from 0.1 up to 24 hours or a few days). The pharmaceutical compositions comprise an oligosaccharide as disclosed herein, or a pharmaceutically acceptable salt, solvate, or prodrug thereof; and one or more release controlling and non-release controlling excipients or carriers, such as those excipients or carriers suitable for a disruptable semi-permeable membrane and as swellable substances.

Disclosed herein also are pharmaceutical compositions in a dosage form for oral administration to a subject, which comprise one or more oligosaccharides (e.g., 3′SL and/or 6′SL and/or 2′FL) as disclosed herein, or a pharmaceutically acceptable salt, solvate, or prodrug thereof; and one or more pharmaceutically acceptable excipients or carriers, enclosed in an intermediate reactive layer comprising a gastric juice-resistant polymeric layered material partially neutralized with alkali and having cation exchange capacity and a gastric juice-resistant outer layer.

Provided herein are pharmaceutical compositions that comprise about 0.1 to about 1000 mg or up to 2000 mg or up to 3000 mg (or any value between 0.1-3000 mg), about 1 to about 500 mg, about 2 to about 100 mg, about 1 mg, about 2 mg, about 3 mg, about 5 mg, about 10 mg, about 15 mg, about 20 mg, about 30 mg, about 40 mg, about 50 mg, about 100 mg, about 500 mg of one or more oligosaccharides as disclosed herein, in the form of immediate release tablets for oral administration. The pharmaceutical compositions further comprise inactive ingredients such as flavoring agents, copovidone, ethylcellulose, magnesium stearate, mannitol, and silicon dioxide.

Provided herein are pharmaceutical compositions that comprise about 0.1 to about 1000 mg or up to 2000 mg or up to 3000 mg (or any value there between), about 1 to about 500 mg, about 2 to about 100 mg, about 1 mg, about 2 mg, about 3 mg, about 5 mg, about 10 mg, about 15 mg, about 20 mg, about 30 mg, about 40 mg, about 50 mg, about 100 mg, about 500 mg of one or more oligosaccharides as disclosed herein, in the form of extended release tablets for oral administration. The pharmaceutical compositions further comprise inactive ingredients such as ethylcellulose, dibutyl sebacate, polyvinyl pyrroliodone, sodium stearyl fumarate, colloidal silicon dioxide, and polyvinylalcohol.

The pharmaceutical compositions disclosed herein may be disclosed in unit-dosage forms or multiple-dosage forms. Unit-dosage forms, as used herein, refer to physically discrete units suitable for administration to human and animal subjects and packaged individually as is known in the art. Each unit-dose contains a predetermined quantity of the oligosaccharide sufficient to produce the desired therapeutic effect, in association with the required pharmaceutical carriers or excipients. Examples of unit-dosage forms include ampoules, syringes, and individually packaged to capsules. Unit-dosage forms may be administered in fractions or multiples thereof. A multiple-dosage form is a plurality of identical unit-dosage forms packaged in a single container to be administered in segregated unit-dosage form. Examples of multiple-dosage forms include vials, bottles of tablets or capsules, or bottles of pints or gallons.

The oligosaccharides as disclosed herein may be administered alone, or in combination with one or more other oligosaccharides disclosed herein, and/or one or more other active ingredients. The pharmaceutical compositions that comprise an oligosaccharide disclosed herein may be formulated in various dosage forms for oral, parenteral, and topical administration. The pharmaceutical compositions may also be formulated as a modified release dosage form, including delayed-, extended-, prolonged-, sustained-, pulsatile-, controlled-, accelerated- and fast-, targeted-, programmed-release, and gastric retention dosage forms.

These dosage forms can be prepared according to conventional methods and techniques known to those skilled in the art (see, Remington: The Science and Practice of Pharmacy, supra; Modified-Release Drug Delivery Technology, Rathbone et al., Eds., Drugs and the Pharmaceutical Science, Marcel Dekker, Inc.: New York, N.Y., 2002; Vol. 126).

The pharmaceutical compositions disclosed herein may be administered at once, or multiple times at intervals of time. It is understood that the precise dosage and duration of treatment may vary with the age, weight, and condition of the patient being treated, and may be determined empirically using known testing protocols or by extrapolation from in vivo or in vitro test or diagnostic data. It is further understood that for any particular individual, specific dosage regimens should be adjusted over time according to the individual need and the professional judgment of the person administering or supervising the administration of the formulations.

Once improvement of the patients condition has occurred, a maintenance dose is administered if necessary. Subsequently, the dosage or the frequency of administration, or both, can be reduced, as a function of the symptoms, to a level at which the improved disease, disorder or condition is retained. Patients can, however, require intermittent treatment on a long-term basis upon any recurrence of symptoms.

The pharmaceutical compositions disclosed herein may be formulated in solid, semisolid, or liquid dosage forms for oral administration. As used herein, oral administration also includes buccal, lingual, and sublingual administration. Suitable oral dosage forms include, but are not limited to, tablets, capsules, pills, troches, lozenges, pastimes, cachets, pellets, medicated chewing gum, granules, bulk powders, effervescent or non-effervescent powders or granules, solutions, emulsions, suspensions, solutions, wafers, sprinkles, elixirs, and syrups. In addition to the oligosaccharides, the pharmaceutical compositions may contain one or more pharmaceutically acceptable carriers or excipients, including, but not limited to, binders, fillers, diluents, disintegrants, wetting agents, lubricants, glidants, coloring agents, dye-migration inhibitors, sweetening agents, and flavoring agents.

Binders or granulators impart cohesiveness to a tablet to ensure the tablet remaining intact after compression. Suitable binders or granulators include, but are not limited to, starches, such as corn starch, potato starch, and pre-gelatinized starch (e.g., STARCH 1500); gelatin; sugars, such as sucrose, glucose, dextrose, molasses, and lactose; natural and synthetic gums, such as acacia, alginic acid, alginates, extract of Irish moss, Panwar gum, ghatti gum, mucilage of isabgol husks, carboxymethyl cellulose, methylcellulose, polyvinylpyrrolidone (PVP), Veegum, larch arabogalactan, powdered tragacanth, and guar gum; celluloses, such as ethyl cellulose, cellulose acetate, carboxymethyl cellulose calcium, sodium carboxymethyl cellulose, methyl cellulose, hydroxyethyl cellulose (HEC), hydroxypropyl cellulose (HPC), hydroxypropyl methyl cellulose (HPMC); microcrystalline celluloses, such as AVICEL-PH-101, AVICEL-PH-103, AVICEL RC-581, AVICEL-PH-105(FMC Corp., Marcus Hook, Pa.); and mixtures thereof. Suitable fillers include, but are not limited to, talc, calcium carbonate, microcrystalline cellulose, powdered cellulose, dextrates, kaolin, mannitol, silicic acid, sorbitol, starch, pre-gelatinized starch, and mixtures thereof. The binder or filler may be present from about 50 to about 99% by weight in the pharmaceutical compositions disclosed herein.

Suitable diluents include, but are not limited to, dicalcium phosphate, calcium sulfate, lactose, sorbitol, sucrose, inositol, cellulose, kaolin, mannitol, sodium chloride, dry starch, and powdered sugar. Certain diluents, such as mannitol, lactose, sorbitol, sucrose, and inositol, when present in sufficient quantity, can impart properties to some compressed tablets that permit disintegration in the mouth by chewing. Such compressed tablets can be used as chewable tablets.

Suitable disintegrants include, but are not limited to, agar; bentonite; celluloses, such as methylcellulose and carboxymethylcellulose; wood products; natural sponge; cation-exchange resins; alginic acid; gums, such as guar gum and Veegum HV; citrus pulp; cross-linked celluloses, such as croscarmellose; cross-linked polymers, such as crospovidone; cross-linked starches; calcium carbonate; microcrystalline cellulose, such as sodium starch glycolate; polacrilin potassium; starches, such as corn starch, potato starch, tapioca starch, and pre-gelatinized starch; clays; aligns; and mixtures thereof. The amount of disintegrant in the pharmaceutical compositions disclosed herein varies upon the type of formulation, and is readily discernible to those of ordinary skill in the art. The pharmaceutical compositions disclosed herein may contain from about 0.5 to about 15% or from about 1 to about 5% by weight of a disintegrant.

It should be understood that many carriers and excipients may serve several functions, even within the same formulation. The pharmaceutical compositions disclosed herein may be formulated as compressed tablets, tablet triturates, chewable lozenges, rapidly dissolving tablets, multiple compressed tablets, or enteric-coating tablets, sugar-coated, or film-coated tablets.

The tablet dosage forms may be prepared from the active ingredient in powdered, crystalline, or granular forms, alone or in combination with one or more carriers or excipients described herein, including binders, disintegrants, controlled-release polymers, lubricants, diluents, and/or colorants. Flavoring and sweetening agents are especially useful in the formation of chewable tablets and lozenges.

The pharmaceutical compositions disclosed herein may be formulated as soft or hard capsules, which can be made from gelatin, methylcellulose, starch, or calcium alginate. The hard gelatin capsule, also known as the dry-filled capsule (DFC), consists of two sections, one slipping over the other, thus completely enclosing the active ingredient. The soft elastic capsule (SEC) is a soft, globular shell, such as a gelatin shell, which is plasticized by the addition of glycerin, sorbitol, or a similar polyol. The soft gelatin shells may contain a preservative to prevent the growth of microorganisms. Suitable preservatives are those as described herein, including methyl- and propyl-parabens, and sorbic acid. The liquid, semisolid, and solid dosage forms disclosed herein may be encapsulated in a capsule. Suitable liquid and semisolid dosage forms include solutions and suspensions in propylene carbonate, vegetable oils, or triglycerides. Capsules containing such solutions can be prepared as described in U.S. Pat. Nos. 4,328,245; 4,409,239; and 4,410,545. The capsules may also be coated as known by those of skill in the art in order to modify or sustain dissolution of the active ingredient.

The pharmaceutical compositions disclosed herein may be formulated in liquid and semisolid dosage forms, including emulsions, solutions, suspensions, elixirs, and syrups. An emulsion is a two-phase system, in which one liquid is dispersed in the form of small globules throughout another liquid, which can be oil-in-water or water-in-oil. Emulsions may include a pharmaceutically acceptable non-aqueous liquids or solvent, emulsifying agent, and preservative.

Suspensions may include a pharmaceutically acceptable suspending agent and preservative. Aqueous alcoholic solutions may include a pharmaceutically acceptable acetal, such as a di(lower alkyl) acetal of a lower alkyl aldehyde (the term “lower” means an alkyl having between 1 and 6 carbon atoms), e.g., acetaldehyde diethyl acetal; and a water-miscible solvent having one or more hydroxyl groups, such as propylene glycol and ethanol. Elixirs are clear, sweetened, and hydroalcoholic solutions. Syrups are concentrated aqueous solutions of a sugar, for example, sucrose, and may also contain a preservative. For a liquid dosage form, for example, a solution in a polyethylene glycol may be diluted with a sufficient quantity of a pharmaceutically acceptable liquid carrier, e.g., water, to be measured conveniently for administration.

Other useful liquid and semisolid dosage forms include, but are not limited to, those containing the active ingredient(s) disclosed herein, and a dialkylated mono- or poly-alkylene glycol.

The pharmaceutical compositions disclosed herein for oral administration may be also formulated in the forms of liposomes, micelles, microspheres, or nanosystems. Micellar dosage forms can be prepared as described in U.S. Pat. No. 6,350,458.

The pharmaceutical compositions disclosed herein may be formulated as non-effervescent or effervescent, granules and powders, to be reconstituted into a liquid dosage form. Pharmaceutically acceptable carriers and excipients used in the non-effervescent granules or powders may include diluents, sweeteners, and wetting agents. Pharmaceutically acceptable carriers and excipients used in the effervescent granules or powders may include organic acids and a source of carbon dioxide.

The pharmaceutical compositions disclosed herein can be formulated as an oral nutritional composition. An oral nutritional composition can contain sources of protein, lipids and/or digestible carbohydrates and can be in solid, powdered or liquid forms. The composition can be designed to be the sole source of nutrition or a nutritional supplement. Suitable protein sources include intact, hydrolyzed, and partially hydrolyzed protein, which can be derived from any suitable source such as milk (e.g., casin, whey), animal (e.g., meat, fish), cereal (e.g., rice, corn), and vegetable (e.g., soy, potato, pea), insect (e.g., locust) and combinations of these sources.

Examples of the source of protein include whey protein concentrates, whey protein isolate, whey protein hydrolysates, and acid.

The pharmaceutical compositions disclosed herein may be formulated as immediate or modified release dosage forms, including delayed-, sustained, pulsed-, controlled, targeted-, and programmed-release forms.

The pharmaceutical compositions disclosed herein may be co-formulated with other active ingredients which do not impair the desired therapeutic action, or with substances that supplement the desired action.

The pharmaceutical compositions disclosed herein may be administered parenterally by injection, infusion, or implantation, for local or systemic administration. Parenteral administration, as used herein, include intravenous, intraarterial, intraperitoneal, intrathecal, intraventricular, intraurethral, intrasternal, intracranial, intramuscular, intrasynovial, and subcutaneous administration.

The pharmaceutical compositions disclosed herein may be formulated in any dosage forms that are suitable for parenteral administration, including solutions, suspensions, emulsions, micelles, liposomes, microspheres, nanosystems, and solid forms suitable for solutions or suspensions in liquid prior to injection. Such dosage forms can be prepared according to conventional methods known to those skilled in the art of pharmaceutical science (see, Remington: The Science and Practice of Pharmacy, supra).

The pharmaceutical compositions intended for parenteral administration may include one or more pharmaceutically acceptable carriers and excipients, including, but not limited to, aqueous vehicles, water-miscible vehicles, non-aqueous vehicles, antimicrobial agents or preservatives against the growth of microorganisms, stabilizers, solubility enhancers, isotonic agents, buffering agents, antioxidants, local anesthetics, suspending and dispersing agents, wetting or emulsifying agents, complexing agents, sequestering or chelating agents, cryoprotectants, lyoprotectants, thickening agents, pH adjusting agents, and inert gases.

The pharmaceutical compositions disclosed herein may be formulated for single or multiple dosage administration. The single dosage formulations are packaged in an ampule, a vial, or a syringe. The multiple dosage parenteral formulations must contain an antimicrobial agent at bacteriostatic or fungistatic concentrations. All parenteral formulations must be sterile, as known and practiced in the art.

The pharmaceutical compositions may be formulated as a suspension, solid, semi-solid, or thixotropic liquid, for administration as an implanted depot. In one embodiment, the pharmaceutical compositions disclosed herein are dispersed in a solid inner matrix, which is surrounded by an outer polymeric membrane that is insoluble in body fluids but allows the active ingredient in the pharmaceutical compositions diffuse through.

Pharmaceutically acceptable carriers and excipients suitable for use in the topical formulations disclosed herein include, but are not limited to, aqueous vehicles, water-miscible vehicles, non-aqueous vehicles, antimicrobial agents or preservatives against the growth of microorganisms, stabilizers, solubility enhancers, isotonic agents, buffering agents, antioxidants, local anesthetics, suspending and dispersing agents, wetting or emulsifying agents, complexing agents, sequestering or chelating agents, penetration enhancers, cryoprotectants, lyoprotectants, thickening agents, and inert gases.

The pharmaceutical compositions disclosed herein may be administered intranasally or by inhalation to the respiratory tract. The pharmaceutical compositions may be formulated in the form of an aerosol or solution for delivery using a pressurized container, pump, spray, atomizer, such as an atomizer using electrohydrodynamics to produce a fine mist, or nebulizer, alone or in combination with a suitable propellant, such as 1,1,1,2-tetrafluoroethane or 1,1,1,2,3,3,3-heptafluoropropane. The pharmaceutical compositions may also be formulated as a dry powder for insufflation, alone or in combination with an inert carrier such as lactose or phospholipids; and nasal drops. For intranasal use, the powder may comprise a bioadhesive agent, including chitosan or cyclodextrin.

The pharmaceutical compositions disclosed herein may be formulated as a modified release dosage form. As used herein, the term “modified release” refers to a dosage form in which the rate or place of release of the active ingredient(s) is different from that of an immediate dosage form when administered by the same route.

Modified release dosage forms include delayed-, extended-, prolonged-, sustained-, pulsatile-, controlled-, accelerated- and fast-, targeted-, programmed-release, and gastric retention dosage forms. The pharmaceutical compositions in modified release dosage forms can be prepared using a variety of modified release devices and methods known to those skilled in the art, including, but not limited to, matrix controlled release devices, osmotic controlled release devices, multiparticulate controlled release devices, ion-exchange resins, enteric coatings, multilayered coatings, microspheres, liposomes, and combinations thereof. The release rate of the active ingredient(s) can also be modified by varying the particle sizes and polymorphism of the active ingredient(s).

The pharmaceutical compositions disclosed herein in a modified release dosage form may be prepared by methods known to those skilled in the art, including direct compression, dry or wet granulation followed by compression, melt-granulation followed by compression.

Generally, the amount of an oligosaccharide disclosed herein required to be administered to the person can vary depending upon factors such as the risk and condition severity, the age of the person, the form of the composition, and other medications being administered to the person. It would be expected that an oligosaccharide described herein should be well tolerated irrespective of the age and condition of the subject. The dosage of oligosaccharide to be administered can readily be set by a medical practitioner and would generally be in the range from about 10 mg to about 20 g per day, in certain embodiments from about 10 mg to about 15 g per day, from about 100 mg to about 10 g per day, in certain embodiments from about 500 mg to about 10 g per day, in certain embodiments from about 1 g to about 7.5 g per day. An appropriate dose can be determined based on several factors, including, for example, the body weight and/or condition of the patient being treated, the severity of the condition, being treated, other ailments and/or diseases of the person, the incidence and/or severity of side effects and the manner of administration. Appropriate dose ranges can be determined by methods known to those skilled in the art. During an initial treatment phase, the dosing can be higher (for example 200 mg to 20 g per day, preferably 500 mg to 15 g per day, more preferably 1 g to 10 g per day, in certain embodiments 2.5 g to 7.5 g per day). During a maintenance phase, the dosing can be reduced (for example, 10 mg to 10 g per day, preferably 100 mg to 7.5 g per day, more preferably 500 mg to 5 g per day, in certain embodiments 1 g to 2.5 g per day).

Depending on the disorder to be treated and the injection in suitable dosage unit with pharmaceutically acceptable carriers, adjuvants and vehicles appropriate for each route of administration.

The dose may be in the form of one, two, three, four, five, six, or more sub-doses that are administered at appropriate intervals per day. The dose or sub-doses can be administered in the form of dosage units containing from about 0.01 to about 2 grams, from about 0.05 to about 1 gram, or from about 10 to about 500 milligrams active ingredient(s) per dosage unit. In certain embodiments, an appropriate dosage level is about 0.01 to about 5 g/kg patient body weight per day (mg/kg per day), about 0.01 to about 1 g/kg per day, about 0.01 to about 0.5 g/kg per day, or about 0.1 to about 500 mg/kg per day, which may be administered in single or multiple doses. A suitable dosage level may be about 0.1 to about 500 mg/kg per day, about 0.1 to about 250 mg/kg per day, or about 0.1 to about 100 mg/kg per day. Within this range the dosage may be about 0.01 to about 0.1, about 0.1 to about 1.0, about 1.0 to about 10, or about 10 to about 100 mg/kg per day.

The oligosaccharides disclosed herein may also be combined or used in combination with other agents useful in the treatment, prevention, or amelioration of one or more symptoms of cancer, as described herein. Or, by way of example only, the therapeutic effectiveness of one of the oligosaccharides described herein may be enhanced by administration of an adjuvant (i.e., by itself the adjuvant may only have minimal therapeutic benefit, but in combination with another therapeutic agent, the overall therapeutic benefit to the patient is enhanced). The HMOs can also be combined or used in combination with other agents to prevent or inhibit progression of the cancer or tumor, improve the efficacy of the immune checkpoint inhibitor(s), reduce the effective dose of the immune checkpoint inhibitor, and/or reduce the toxicity of the immune checkpoint inhibitor in a subject. The HMOs can also be combined or used in combination with other agents to prevent or delay the incidence of cancer in a subject, for example, a subject at risk of cancer.

Such other agents, adjuvants, or drugs may be administered, by a route and in an amount commonly used therefore, simultaneously or sequentially with an oligosaccharide as disclosed herein. When an oligosaccharide as disclosed herein is used contemporaneously with one or more other drugs, a pharmaceutical composition containing such other drugs in addition to an oligosaccharide disclosed herein may be utilized, but is not required.

Accordingly, the pharmaceutical compositions disclosed herein include those that also contain one or more other active ingredients or therapeutic agents, in addition to an oligosaccharide disclosed herein.

### EXAMPLES

The compositions and methods of the invention can be evaluated for in vivo efficacy in one or more mouse models described in Zitvogel, L., Pitt, J., Daillère, R. et al. Mouse models in oncoimmunology. Nat Rev Cancer 16, 759-773 (2016). https://doi.org/10.1038/nrc.2016.91; the contents of which are expressly incorporated by reference herein.

While this invention has been particularly shown and described with references to preferred embodiments thereof, it will be understood by those skilled in the art that various changes in form and details may be made therein without departing from the scope of the invention encompassed by the appended claims.

1. A pharmaceutical composition comprising a human milk oligosaccharide selected from the group consisting of 2′fucosyllactose, 3′ sialyllactose, and 6′ sialyllactose, wherein the composition further comprises an immune checkpoint inhibitor.

2. The composition of claim 1, wherein the immune checkpoint inhibitor is an inhibitor of PD-1, PD-L1, PD-L2, PD-L3, PD-L4, CTLA-4, LAG3, B7-H3, B7-H4, KIR or TIM3.

3. The composition of claim 2, wherein the immune checkpoint inhibitor is a PD-1 inhibitor, a PD-L1 inhibitor, a PD-L2 inhibitor, or a CTLA-4 inhibitor.

4. (canceled)

5. (canceled)

6. (canceled)

7. The composition of claim 1, comprising a first immune checkpoint inhibitor and a second immune checkpoint inhibitor, wherein the first immune checkpoint inhibitor is different from the second immune checkpoint inhibitor.

8. The composition of claim 7, wherein the first and the second immune checkpoint inhibitor are each independently an inhibitor of PD-1, PD-L1, PD-L2, PD-L3, PD-L4, CTLA-4, LAG3, B7-H3, B7-H4, KIR or TIM3.

9. (canceled)

10. (canceled)

11. (canceled)

12. The composition of claim 1, wherein the immune checkpoint inhibitor is an antibody.

13. (canceled)

14. (canceled)

15. (canceled)

16. (canceled)

17. (canceled)

18. The composition of claim 1, wherein the composition comprises a combination of any one of 2′fucosyllactose, 3′ sialyllactose, and 6′ sialyllactose.

19. The composition of claim 18, wherein the composition comprises a combination of 2′fucosyllactose, 3′ sialyllactose and 6′ sialyllactose.

20. The composition of claim 1, further comprising one or more pharmaceutically acceptable excipients.

21. The composition of claim 1, further comprising one or more additional chemotherapeutic agent.

22. The composition of claim 1, wherein the immune checkpoint inhibitor is nivolumab, pembrolizumab, pidilizumab, ipilimumab, dacarbazine, BMS 936559, atezolizumab, durvalumab, or any combination thereof.

23. A method for treating cancer, comprising administering the pharmaceutical composition of claim 1 to a subject in need thereof.

24. A method for treating cancer, comprising co-administering an effective amount of a human milk oligosaccharide and an effective amount of one or more immune checkpoint inhibitors to a subject in need thereof.

25. The method of claim 24, wherein the human milk oligosaccharide is selected from the group consisting of 2′fucosyllactose, 3′ sialyllactose, and 6′ sialyllactose.

26. The method of claim 25, wherein the human milk oligosaccharide is a combination of any two of 2′fucosyllactose, 3′ sialyllactose and 6′ sialyllactose.

27. The method of claim 25, wherein the human milk oligosaccharide is a combination of 2′fucosyllactose, 3′ sialyllactose and 6′ sialyllactose.

28. The method of claim 24, further comprising co-administering an effective amount of an additional chemotherapeutic agents.

29. The method of claim 24, wherein the cancer comprises cancer cells expressing a binding ligand of PD-1, PD-L1 or PD-L2.

30. (canceled)

31. The method of claim 29, wherein the cancer is head and neck cancer, lung cancer, stomach cancer, colon cancer, pancreatic cancer, prostate cancer, breast cancer, kidney cancer, bladder cancer, ovary cancer, cervical cancer, melanoma, glioblastoma, myeloma, lymphoma, or leukemia.

32. The method of claim 29, wherein the cancer is renal cell carcinoma, malignant melanoma, non-small cell lung cancer (NSCLC), ovarian cancer, Hodgkin's lymphoma or squamous cell carcinoma.

33. The method of claim 24, wherein the cancer comprises cancer cells expressing a binding ligand of CTLA-4.

34. The method of claim 33, wherein the binding ligand is B7.1 or B7.2.

35. The method of claim 24, wherein the immune checkpoint inhibitor is an inhibitor of PD-1, PD-L1, PD-L2, PD-L3, PD-L4, CTLA-4, LAG3, B7-H3, B7-H4, KIR or TIM3.

36. The method of claim 35, wherein the immune checkpoint inhibitor is a PD-1 inhibitor, a PD-L1 inhibitor, a PD-L2 inhibitor or a CTLA inhibitor.

37. (canceled)

38. (canceled)

39. (canceled)

40. The method of claim 24, comprising a first immune checkpoint inhibitor and a second immune checkpoint inhibitor, wherein the first immune checkpoint inhibitor is different from the second immune checkpoint inhibitor.

41. The method of claim 40, wherein the first and the second immune checkpoint inhibitor are each independently an inhibitor of PD-1, PD-L1, PD-L2, PD-L3, PD-L4, CTLA-4, LAG3, B7-H3, B7-H4, KIR or TIM3.

42. The method of claim 41, wherein the first immune checkpoint inhibitor is a PD-1 inhibitor, and the second immune checkpoint inhibitor is a CTLA-4 inhibitor.

43. The method of claim 24, wherein the immune checkpoint inhibitor is an antibody.

44. (canceled)

45. (canceled)

46. (canceled)

47. (canceled)

48. (canceled)

49. (canceled)

50. The method of claim 24, wherein the cancer is selected from breast cancer, colon cancer, rectal cancer, lung cancer, prostate cancer, melanoma, leukemia, ovarian cancer, gastric cancer, renal cell carcinoma, liver cancer, pancreatic cancer, lymphomas and myeloma.

51. The method of claim 24, wherein the cancer is a solid tumor or hematological cancer.

52. The method of claim 24, wherein the cancer does not have any cells expressing PD-1, PD-L1, or PD-L2.

53. The method of claim 24, wherein the human milk oligosaccharide is administered to the subject before, during or after beginning immune checkpoint inhibitor treatment.
